(19) United States
(12) Patent Application Publication
(10) Pub. No.: US 2010/0135960 A1
(43) Pub. Date: Jun. 3, 2010

(54) ANTIVIRAL ACYLSULFONAMIDE DERIVATIVES

(76) Inventors: Yat Sun Or, Watertown, MA (US); Lu Ying, Belmont, MA (US); Ce Wang, Waltham, MA (US); Xiaowen Peng, Auburndale, MA (US); Yao-Ling Qiu, Andover, MA (US)

Correspondence Address: ELMORE PATENT LAW GROUP, PC, 515 Groton Road, Unit 1R, Westford, MA, 01886, US

(21) Appl. No.: 12/619,974

(22) Filed: Nov. 17, 2009

Related U.S. Application Data
(60) Provisional application No. 61/115,749, filed on Nov. 18, 2008.

(51) Int. Cl. A61K 38/21; A61K 39/00; A61K 38/19; A61K 31/4439; A61K 31/427; A61K 31/422; C07D 401/02; C07D 417/02; C07D 413/02; A61P 31/14

(52) U.S. Cl. 424/85.5; 424/85.6; 424/85.7; 424/184.1; 514/12; 514/341; 514/370; 514/380; 546/275.4; 548/195; 548/245

(57) ABSTRACT

The present invention discloses compounds of Formula (I), or pharmaceutically acceptable salts, esters, or prodrugs thereof:

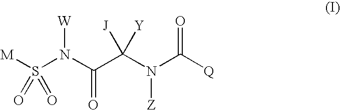

which inhibit RNA-containing virus, particularly the hepatitis C virus (HCV). Consequently, the compounds of the present invention interfere with the life cycle of the hepatitis C virus and are also useful as antiviral agents. The present invention further relates to pharmaceutical compositions comprising the aforementioned compounds for administration to a subject suffering from HCV infection. The invention also relates to methods of treating an HCV infection in a subject by administering a pharmaceutical composition comprising the compounds of the present invention. The present invention relates to novel antiviral compounds represented herein above, pharmaceutical compositions comprising such compounds, and methods for the treatment or prophylaxis of viral (particularly HCV) infection in a subject in need of such therapy with said compounds.

### RELATED APPLICATION

This application claims the benefit of U.S. Provisional Application No. 61/115,749, filed on Nov. 18, 2008. The entire teachings of the above application(s) are incorporated herein by reference.

### TECHNICAL FIELD

The present invention relates to novel anti-infective agents. Specifically, the present invention relates to compounds, compositions, a method for inhibiting hepatitis C virus (HCV) polymerase, a method for inhibiting HCV viral replication, and a method for treating or preventing HCV infection.

### BACKGROUND OF THE INVENTION

Infection with HCV is a major cause of human liver disease throughout the world. In the US, an estimated 4.5 million Americans are chronically infected with HCV. Although only 30% of acute infections are symptomatic, greater than 85% of infected individuals develop chronic, persistent infection. Treatment costs for HCV infection have been estimated at $5.46 billion for the US in 1997. Worldwide over 200 million people are estimated to be infected chronically. HCV infection is responsible for 40-60% of all chronic liver disease and 30% of all liver transplants. Chronic HCV infection accounts for 30% of all cirrhosis, end-stage liver disease, and liver cancer in the U.S. The CDC estimates that the number of deaths due to HCV will minimally increase to 38,000/year by the year 2010.

Due to the high degree of variability in the viral surface antigens, existence of multiple viral genotypes, and demonstrated specificity of immunity, the development of a successful vaccine in the near future is unlikely. Alpha-interferon (alone or in combination with ribavirin) has been widely used since its approval for treatment of chronic HCV infection. However, adverse side effects are commonly associated with this treatment: flu-like symptoms, leukopenia, thrombocytopenia, depression from interferon, as well as anemia induced by ribavirin (Lindsay, K. L. (1997) Hepatology 26 (suppl 1): 71S-77S). This therapy remains less effective against infections caused by HCV genotype 1 (which constitutes ˜75% of all HCV infections in the developed markets) compared to infections caused by the other 5 major HCV genotypes. Unfortunately, only ˜50-80% of the patients respond to this treatment (measured by a reduction in serum HCV RNA levels and normalization of liver enzymes) and, of responders, 50-70% relapse within 6 months of cessation of treatment. Recently, with the introduction of pegylated interferon (Peg-IFN), both initial and sustained response rates have improved substantially, and combination treatment of Peg-IFN with ribavirin constitutes the gold standard for therapy. However, the side effects associated with combination therapy and the impaired response in patients with genotype 1 present opportunities for improvement in the management of this disease.

First identified by molecular cloning in 1989 (Choo, Q-L et al (1989) Science 244:359-362), HCV is now widely accepted as the most common causative agent of post-transfusion non-A, non-B hepatitis (NANBH) (Kuo, G et al (1989) Science 244:362-364). Due to its genome structure and sequence homology, this virus was assigned as a new genus in the Flaviviridae family. Like the other members of the Flaviviridae, such as flaviviruses (e.g. yellow fever virus and Dengue virus types 1-4) and pestiviruses (e.g. bovine viral diarrhea virus, border disease virus, and classic swine fever virus) (Choo, Q-L et al (1989) Science 244:359-362; Miller, R. H. and R. H. Purcell (1990) Proc. Natl. Acad. Sci. USA 87:2057-2061), HCV is an enveloped virus containing a single strand RNA molecule of positive polarity. The HCV genome is approximately 9.6 kilobases (kb) with a long, highly conserved, noncapped 5′ nontranslated region (NTR) of approximately 340 bases which functions as an internal ribosome entry site (IRES) (Wang C Y et al ‘An RNA pseudoknot is an essential structural element of the internal ribosome entry site located within the hepatitis C virus 5′ noncoding region’ RNA A Publication of the RNA Society. 1(5): 526-537, 1995 July). This element is followed by a region which encodes a single long open reading frame (ORF) encoding a polypeptide of ˜3000 amino acids comprising both the structural and nonstructural viral proteins.

Upon entry into the cytoplasm of the cell, this RNA is directly translated into a polypeptide of ˜3000 amino acids comprising both the structural and nonstructural viral proteins. This large polypeptide is subsequently processed into the individual structural and nonstructural proteins by a combination of host and virally-encoded proteinases (Rice, C. M. (1996) in B. N. Fields, D. M. Knipe and P. M. Howley (eds) Virology 2^{nd} Edition, p 931-960; Raven Press, N. Y.). There are three structural proteins, C, E1 and E2. The P7 protein is of unknown function and is comprised of a highly variable sequence. There are six non-structural proteins. NS2 is a zinc-dependent metalloproteinase that functions in conjunction with a portion of the NS3 protein. NS3 incorporates two catalytic functions (separate from its association with NS2): a serine protease at the N-terminal end, which requires NS4A as a cofactor, and an ATP-ase-dependent helicase function at the carboxyl terminus. NS4A is a tightly associated but non-covalent cofactor of the serine protease. NS5A is a membrane-anchored phosphoprotein that is observed in basally phosphorylated (56 kDa) and hyperphosphorylated (58 kDa) forms. While its function has not fully been elucidated, NS5A is believed to be important in viral replication.

Following the termination codon at the end of the long ORF, there is a 3′ NTR which roughly consists of three regions: an ˜40 base region which is poorly conserved among various genotypes, a variable length poly(U)/polypyrimidine tract, and a highly conserved 98 base element also called the “3′ X-tail” (Kolykhalov, A. et al (1996) J. Virology 70:3363-3371; Tanaka, T. et al (1995) Biochem Biophys. Res. Commun. 215744-749; Tanaka, T. et al (1996) J. Virology 70:3307-3312; Yamada, N. et al (1996) Virology 223:255-261). The 3′ NTR is predicted to form a stable secondary structure which is essential for HCV growth in chimps and is believed to function in the initiation and regulation of viral RNA replication.

The NS5B protein (591 amino acids, 65 kDa) of HCV (Behrens, S. E. et al (1996) EMBO J. 151 2-22), encodes an RNA-dependent RNA polymerase (RdRp) activity and contains canonical motifs present in other RNA viral polymerases. The NS5B protein is fairly well conserved both intra-typically (˜95-98% amino acid (aa) identity across 1b isolates) and inter-typically (˜85% aa identity between genotype 1a and 1b isolates). The essentiality of the HCV NS5B RdRp activity for the generation of infectious progeny virions has been formally proven in chimpanzees (A. A. Kolykhalov et al. (2000) Journal of Virology, 74(4): 2046-2051). Thus, inhibition of NS5B RdRp activity (inhibition of RNA replication) is predicted to be useful to treat HCV infection.

Based on the foregoing, there exists a significant need to identify compounds with the ability to inhibit HCV. A general strategy for the development of antiviral agents is to inactivate virally encoded enzymes, including NS5B, that are essential for the replication of the virus.

### SUMMARY OF THE INVENTION

The present invention relates to novel antiviral compounds represented herein below, pharmaceutical compositions comprising such compounds, and methods for the treatment or prophylaxis of viral (particularly HCV) infection in a subject in need of such therapy with said compounds.

In its principal aspect, the present invention provides a compound of formula

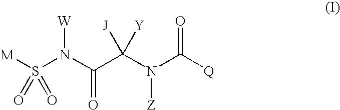

or a pharmaceutically acceptable salt, ester, stereoisomer, tautomer, solvate, prodrug, or combination thereof, in combination with a pharmaceutically acceptable carrier or excipient, wherein:

M is —R₁ or —NR₂R₂ₐ; wherein R₂ and R₂ₐ at each occurrence are each independently hydrogen or —R₁; or R₂ and R₂ₐ taken together with the nitrogen atom to which they are attached form an optionally substituted heterocyclic or optionally substituted heteroaryl group; and R₁ at each occurrence is independently selected from the group consisting of: optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted heterocyclic, optionally substituted aryl and optionally substituted heteroaryl;

W is hydrogen or hydroxy;

Q is an optionally substituted aryl or optionally substituted heteroaryl, preferably 4-tent-butyl-3-methoxyphenyl or 4-tent-butyl-3-halophenyl or 5-tert-butyl-4-methoxypyridin-2-yl;

Y is selected from the group consisting of: optionally substituted —C₁-C₈ alkyl, optionally substituted —C₃-C₆ alkenyl or optionally substituted —C₃-C₆ alkynyl each containing 0, 1, 2, or 3 heteroatoms selected from O, S or N;

J is —C₁-C₄ alkyl substituted with —O—C₁-C₄ alkyl, —N(—C₁-C₄ alkyl)₂, optionally substituted heterocyclic, optionally substituted aryl or optionally substituted heteroaryl, preferably 1H-pyrazol-1-ylmethyl or 1,3-thiazol-4-ylmethyl; and

Z is a —C₁-C₄ alkyl substituted with an optionally substituted heterocyclic, optionally substituted aryl or optionally substituted heteroaryl, preferably thiazol-2-ylmethyl or thiazol-2-ylethyl or (5-methyl-isoxazol-3-yl)methyl or benzyl.

Each preferred group stated above can be taken in combination with one, any or all other preferred groups.

In another aspect, the present invention provides a pharmaceutical composition comprising a therapeutically effective amount of a compound or combination of compounds of the present invention, or a pharmaceutically acceptable salt form, prodrug, salt of a prodrug, stereoisomer, tautomer, solvate, or combination thereof, in combination with a pharmaceutically acceptable carrier or excipient.

In yet another aspect, the present invention provides a method of inhibiting the replication of a RNA-containing virus comprising contacting said virus with a therapeutically effective amount of a compound or a combination of compounds of the present invention, or a pharmaceutically acceptable salt, prodrug, salt of a prodrug, stereoisomer, tautomer, solvate, or combination thereof. Particularly, this invention is directed to methods of inhibiting the replication of hepatitis C virus.

In still another aspect, the present invention provides a method of treating or preventing infection caused by an RNA-containing virus comprising administering to a patient in need of such treatment a therapeutically effective amount of a compound or combination of compounds of the present invention, or a pharmaceutically acceptable salt form, prodrug, salt of a prodrug, stereoisomer, or tautomer, solvate, or combination thereof. Particularly, this invention is directed to methods of treating or preventing infection caused by hepatitis C virus.

Yet another aspect of the present invention provides the use of a compound or combination of compounds of the present invention, or a therapeutically acceptable salt form, prodrug, salt of a prodrug, stereoisomer or tautomer, solvate, or combination thereof, as defined hereinafter, in the preparation of a medicament for the treatment or prevention of infection caused by RNA-containing virus, specifically hepatitis C virus (HCV).

### DETAILED DESCRIPTION OF THE INVENTION

In an embodiment of the present invention is a compound of Formula (I) as illustrated above, or a pharmaceutically acceptable salt, ester or prodrug thereof.

In an embodiment of the present invention, the absolute stereochemistry of a racemic compound of Formula (I), is represented by Formula (Ia):

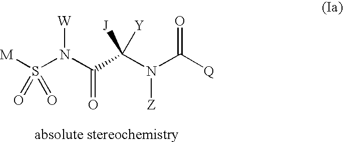

wherein M, W, Q, Y, Z and J are as previously defined.

In an embodiment, the present invention relates to a compound of Formula (IIa), or a pharmaceutically acceptable salt, ester or prodrug thereof:

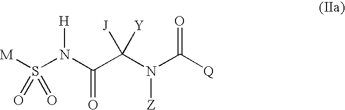

wherein M, Q, Z, Y and J are as previously defined.

In an embodiment, the present invention relates to a compound of Formula (IIb), or a pharmaceutically acceptable salt, ester or prodrug thereof:

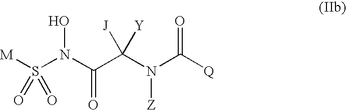

wherein M, Q, Z, Y and J are as previously defined.

In an embodiment, the present invention relates to a compound of Formula (IIe), or a pharmaceutically acceptable salt, ester or prodrug thereof:

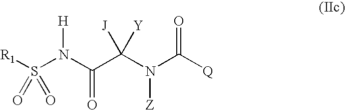

wherein R₁, Q, Z, Y and J are as previously defined.

In an embodiment, the present invention relates to a compound of Formula (IId), or a pharmaceutically acceptable salt, ester or prodrug thereof:

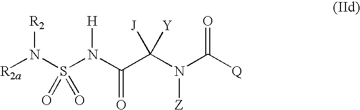

wherein R₂, R₂ₐ, Q, Z, Y and J are as previously defined.

In an embodiment, the present invention relates to a compound of Formula (IIe), or a pharmaceutically acceptable salt, ester or prodrug thereof:

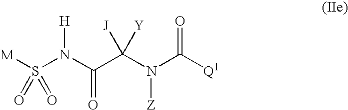

wherein M, Z, Y and J are as previously defined and Q¹ is a substituted aryl, preferably 4-tent-butyl-3-methoxyphenyl or 4-tent-butyl-3-halophenyl.

In an embodiment, the present invention relates to a compound of Formula (IIf), or a pharmaceutically acceptable salt, ester or prodrug thereof:

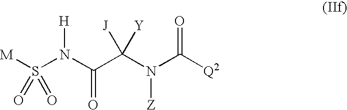

wherein M, Z, Y and J are as previously defined and Q² is a substituted heteroaryl, preferably 5-tent-butyl-4-methoxypyridin-2-yl.

In an embodiment, the present invention relates to a compound of Formula (IIg), or a pharmaceutically acceptable salt, ester or prodrug thereof:

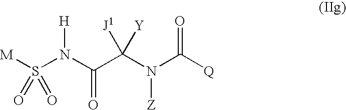

wherein M, Q, Z and Y are as previously defined and J¹ is a methyl or ethyl substituted with optionally substituted aryl or optionally substituted heteroaryl, preferably 1H-pyrazol-1-ylmethyl or 1,3-thiazol-4-ylmethyl.

In an embodiment, the present invention relates to a compound of Formula (IIh), or a pharmaceutically acceptable salt, ester or prodrug thereof:

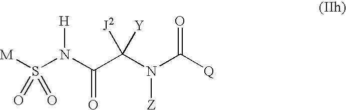

wherein M, Q, Z and Y are as previously defined and J² is a —C₁-C₄ alkyl substituted with —O—C₁-C₄ alkyl, —N(—C₁-C₄ alkyl)₂ or optionally substituted heterocyclic.

In an embodiment, the present invention relates to a compound of Formula (IIi), or a pharmaceutically acceptable salt, ester or prodrug thereof:

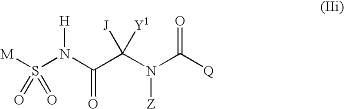

wherein M, Q, Z and J are as previously defined and Y¹ is independently a substituted —C₁-C₄ alkyl containing 0, 1, 2, or 3 heteroatoms selected from O, S or N.

In an embodiment, the present invention relates to a compound of Formula (IIj), or a pharmaceutically acceptable salt, ester or prodrug thereof:

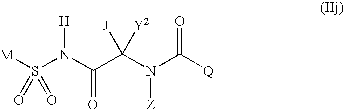

wherein M, Q, Z and J are as previously defined and Y² is independently an optionally substituted —C₃-C₆ alkenyl containing 0, 1, 2, or 3 heteroatoms selected from O, S or N.

Representative compounds of the present invention are those selected from:

1. Compound of Formula (I), wherein M=Me, Q=4-tent-butyl-3-methoxyphenyl, Z=thiazol-2-ylmethyl, W═H, Y=—CH₂CH═CH₂, J=1H-pyrazol-1-ylmethyl.
2. Compound of Formula (I), wherein M=Me, Q=4-tent-butyl-3-methoxyphenyl, Z=thiazol-2-ylmethyl, W═H, Y=—CH₂CH₂CH₂OMe, J=1H-pyrazol-1-ylmethyl.
3. Compound of Formula (I), wherein M=Me, Q=4-tent-butyl-3-methoxyphenyl, Z=thiazol-2-ylmethyl, W═H, Y=—CH₂CH₂CH═NOMe, J=1H-pyrazol-1-ylmethyl.
4. Compound of Formula (I), wherein M=Me, Q=4-tent-butyl-3-methoxyphenyl, Z=thiazol-2-ylmethyl, W═H, Y=—CH₂CH₂CH₂NMe₂, J=1H-pyrazol-1-ylmethyl.
5. Compound of Formula (I), wherein M=Me, Q=4-tent-butyl-3-methoxyphenyl, Z=thiazol-2-ylmethyl, W═H, Y=(E)-CH₂CH₂CH═CHCN, J=1H-pyrazol-1-ylmethyl.
6. Compound of Formula (I), wherein M=Me, Q=4-tent-butyl-3-methoxyphenyl, Z=thiazol-2-ylmethyl, W═H, Y=(Z)-CH₂CH₂CH═CHCN, J=1H-pyrazol-1-ylmethyl.
7. Compound of Formula (I), wherein M=Me, Q=4-tent-butyl-3-methoxyphenyl, Z=thiazol-2-ylmethyl, W═H, Y=—CH₂CH₂CH₂CH₂CN, J=1H-pyrazol-1-ylmethyl.
8. Compound of Formula (I), wherein M=Me, Q=4-tent-butyl-3-methoxyphenyl, Z=thiazol-2-ylmethyl, W═H, Y=—CH₂CH₂CH═CF₂, J=1H-pyrazol-1-ylmethyl.
9. Compound of Formula (I), wherein M=Me, Q=4-tent-butyl-3-methoxyphenyl, Z=thiazol-2-ylmethyl, W═H, Y=—CH₂CHO, J=1H-pyrazol-1-ylmethyl.
10. Compound of Formula (I), wherein M=Me, Q=4-tent-butyl-3-methoxyphenyl, Z=thiazol-2-ylmethyl, W═H, Y=—CH₂CH₂OMe, J=1H-pyrazol-1-ylmethyl.
11. Compound of Formula (I), wherein M=Me, Q=4-tent-butyl-3-methoxyphenyl, Z=thiazol-2-ylmethyl, W═H, Y=(E)-CH₂CH═CHCN, J=1H-pyrazol-1-ylmethyl.
12. Compound of Formula (I), wherein M=Me, Q=4-tent-butyl-3-methoxyphenyl, Z=thiazol-2-ylmethyl, W═H, Y=(Z)-CH₂CH═CHCN, J=1H-pyrazol-1-ylmethyl.
13. Compound of Formula (I), wherein M=Me, Q=4-tent-butyl-3-methoxyphenyl, Z=thiazol-2-ylmethyl, W═H, Y=(E)-CH₂CH₂CH₂CN, J=1H-pyrazol-1-ylmethyl.
14. Compound of Formula (I), wherein M=Me, Q=4-tent-butyl-3-methoxyphenyl, Z=thiazol-2-ylmethyl, W═H, Y=—CH₂CH═NOMe, J=1H-pyrazol-1-ylmethyl.
15. Compound of Formula (I), wherein M=Me, Q=4-tent-butyl-3-methoxyphenyl, Z=thiazol-2-ylmethyl, W═H, Y=—CH₂CH₂NMe₂, J=1H-pyrazol-1-ylmethyl.
16. Compound of Formula (I), wherein M=Me, Q=4-tent-butyl-3-methoxyphenyl, Z=thiazol-2-ylmethyl, W═H, Y=—CH₂CH═CH₂, J=1H-pyrazol-1-ylmethyl.
17. Compound of Formula (I), wherein M=Me, Q=4-tent-butyl-3-methoxyphenyl, Z=thiazol-2-ylmethyl, W═H, Y=—CH₂CH═CH₂, J=1H-pyrazol-1-ylmethyl.
18. Compound of Formula (I), wherein M=Ph, Q=4-tent-butyl-3-methoxyphenyl, Z=thiazol-2-yl, W═H, Y=—CH₂CH₂CN, J=1H-pyrazol-1-ylmethyl.
19. Compound of Formula (I), wherein M=Ph, Q=4-tent-butyl-3-methoxyphenyl, Z=thiazol-2-yl, W═H, Y=—CH₂CN, J=1H-pyrazol-1-ylmethyl.
20. Compound of Formula (I), wherein M=-Ph, Q=4-tent-butyl-3-methoxyphenyl, Z=thiazol-2-yl, W═H, Y=—CH₂CH₂-(tetrazol-5-yl), J=1H-pyrazol-1-ylmethyl.
21. Compound of Formula (I), wherein M=-Ph, Q=4-tent-butyl-3-methoxyphenyl, Z=thiazol-2-yl, W═H, Y=—CH₂-(tetrazol-5-yl), J=1H-pyrazol-1-ylmethyl.
22. Compound of Formula (I), wherein M=—NH₂, Q=4-tent-butyl-3-methoxyphenyl, Z=thiazol-2-ylmethyl, W═H, Y=—CH₂CH₂CH₂OMe, J=1H-pyrazol-1-ylmethyl.
23. Compound of Formula (I), wherein M=—NHPh, Q=4-tent-butyl-3-methoxyphenyl, Z=thiazol-2-ylmethyl, W═H, Y=—CH₂CH₂CH₂OMe, J=1H-pyrazol-1-ylmethyl.
24. Compound of Formula (I), wherein M=—NMe₂, Q=4-tent-butyl-3-methoxyphenyl, Z=thiazol-2-ylmethyl, W═H, Y=—CH₂CH₂CH₂OMe, J=1H-pyrazol-1-ylmethyl.
25. Compound of Formula (I), wherein M=-Ph, Q=4-tent-butyl-3-methoxyphenyl, Z=thiazol-2-ylmethyl, W═OH, Y=—CH₂CH₂CH₂OMe, J=1H-pyrazol-1-ylmethyl.
26. Compound of Formula (I), wherein M=-Phenyl-fluoro-(p), Q=4-tent-butyl-3-methoxyphenyl, Z=thiazol-2-ylmethyl, W═H, Y=—CH₂CH₂CH₂OMe, J=1H-pyrazol-1-ylmethyl.
27. Compound of Formula (I), wherein M=Phenyl-fluoro-(m), Q=4-tent-butyl-3-methoxyphenyl, Z=thiazol-2-ylmethyl, W═H, Y=—CH₂CH₂CH₂OMe, J=1H-pyrazol-1-ylmethyl.
28. Compound of Formula (I), wherein M=-Phenyl-fluoro-(O), Q=4-tent-butyl-3-methoxyphenyl, Z=thiazol-2-ylmethyl, W═H, Y=—CH₂CH₂CH₂OMe, J=1H-pyrazol-1-ylmethyl.
29. Compound of Formula (I), wherein M=-2-pyridyl, Q=4-tent-butyl-3-methoxyphenyl, Z=thiazol-2-ylmethyl, W═H, Y=—CH₂CH₂CH₂OMe, J=1H-pyrazol-1-ylmethyl.
30. Compound of Formula (I), wherein M=-3-pyridyl, Q=4-tent-butyl-3-methoxyphenyl, Z=thiazol-2-ylmethyl, W═H, Y=—CH₂CH₂CH₂OMe, J=1H-pyrazol-1-ylmethyl.
31. Compound of Formula (I), wherein M=-4-pyridiyl, Q=4-tert-butyl-3-methoxyphenyl, Z=thiazol-2-ylmethyl, W═H, Y=—CH₂CH₂CH₂OMe, J=1H-pyrazol-1-ylmethyl.
32. Compound of Formula (I), wherein M=-Ph, Q=4-tent-butyl-3-trifluoromethoxyphenyl, Z=thiazol-2-ylmethyl, W═H, Y=—CH₂CH₂CH₂OMe, J=1H-pyrazol-1-ylmethyl.
33. Compound of Formula (I), wherein M=-Ph, Q=5-tert-butyl-4-methoxypyridin-2-yl, Z=thiazol-2-ylmethyl, W═H, Y=—CH₂CH₂CH₂OMe, J=1H-pyrazol-1-ylmethyl.
34. Compound of Formula (I), wherein M=-Ph, Q=4-tent-butyl-3-vinylphenyl, Z=thiazol-2-ylmethyl, W═H, Y=—CH₂CH₂CH₂OMe, J=1H-pyrazol-1-ylmethyl.
35. Compound of Formula (I), wherein M=-Ph, Q=4-tent-butyl-3-bromophenyl, Z=thiazol-2-ylmethyl, W═H, Y=—CH₂CH₂CH₂OMe, J=1H-pyrazol-1-ylmethyl.
36. Compound of Formula (I), wherein M=-Ph, Q=4-tent-butyl-3-chlorophenyl, Z=thiazol-2-ylmethyl, W═H, Y=—CH₂CH₂CH₂OMe, J=1H-pyrazol-1-ylmethyl.
37. Compound of Formula (I), wherein M=-Ph, Q=4-tent-butyl-3-fluorophenyl, Z=thiazol-2-ylmethyl, W═H, Y=—CH₂CH₂CH₂OMe, J=1H-pyrazol-1-ylmethyl.
38. Compound of Formula (I), wherein M=-Ph, Q=4-tert-butyl-5-bromo-2-fluorophenyl, Z=thiazol-2-ylmethyl, W═H, Y=—CH₂CH₂CH₂OMe, J=1H-pyrazol-1-ylmethyl.
39. Compound of Formula (I), wherein M=-Ph, Q=4-tert-butyl-5-bromo-2-fluorophenyl, Z=(5-methyl-isoxazol-3-yl)methyl, W═H, Y=—CH₂CH₂CH₂OMe, J=1H-pyrazol-1-ylmethyl.
40. Compound of Formula (I), wherein M=-Ph, Q=4-tert-butyl-5-bromo-2-fluorophenyl, Z=thiazol-2-ylmethyl, W═H, Y=—CH₂CH₂CH₂OMe, J=1,3-thiazol-4-ylmethyl.
41. Compound of Formula (I), wherein M=-Ph, Q=4-tert-butyl-5-bromo-2-fluorophenyl, Z=thiazol-2-ylmethyl, W═H, Y=—CH₂CH₂CH₂OMe, J=1,3-thiazol-2-ylmethyl.
42. Compound of Formula (I), wherein M=-Ph, Q=4-tert-butyl-5-bromo-2-fluorophenyl, Z=thiazol-2-ylmethyl, W═H, Y=—CH₂CH₂CH₂OMe, J=isothiazol-3-ylmethyl.
43. Compound of Formula (I), wherein M=-Ph, Q=4-tert-butyl-5-bromo-2-fluorophenyl, Z=Bn, W═H, Y=—CH₂CH₂CH₂OMe, J=isothiazol-3-ylmethyl.
44. Compound of Formula (I), wherein M=-Ph, Q=4-tert-butyl-5-bromo-2-fluorophenyl, Z=thiazol-2-ylmethyl, W═H, Y=—CH₂CH₂CH₂OMe, J=Bn.
45. Compound of Formula (I), wherein M=-Ph, Q=4-tent-butyl-3-methoxyphenyl, Z=thiazol-2-ylmethyl, W═H, Y=propargyl, J=1H-pyrazol-1-ylmethyl.
46. Compound of Formula (I), wherein M=-Ph, Q=4-tent-butyl-3-methoxyphenyl, Z=thiazol-2-ylmethyl, W═H, Y=—CH₂CH₂CH₂OMe, J=—CH₂CH₂OMe.
47. Compound of Formula (I), wherein M=-Ph, Q=4-tent-butyl-3-methoxyphenyl, Z=thiazol-2-ylmethyl, W═H, Y=—CH₂CH₂CH₂OMe, J=—CH₂CH₂NMe₂.
48. Compound of Formula (I), wherein M=-Ph, Q=4-tent-butyl-3-methoxyphenyl, Z=2-(thiazol-2-yl)ethyl, W═H, Y=—CH₂CH₂CH₂OMe, J=—CH₂CH₂NMe₂.
49. Compound of Formula (I), wherein M=2,4-difluorophenyl, Q=4-tent-butyl-3-methoxyphenyl, Z=thiazol-2-ylmethyl, W═H, Y=—CH₂CH₂CH₂OMe, J=1H-pyrazol-1-ylmethyl.
50. Compound of Formula (I), wherein M=tert-butyl, Q=4-tent-butyl-3-methoxyphenyl, Z=thiazol-2-ylmethyl, W═H, Y=—CH₂CH₂CH₂OMe, J=1H-pyrazol-1-ylmethyl.
51. Compound of Formula (I), wherein M=cyclopropyl, Q=4-tent-butyl-3-methoxyphenyl, Z=thiazol-2-ylmethyl, W═H, Y=—CH₂CH₂CH₂OMe, J=1H-pyrazol-1-ylmethyl.
52. Compound of Formula (I), wherein M=trifluoromethyl, Q=4-tent-butyl-3-methoxyphenyl, Z=thiazol-2-ylmethyl, W═H, Y=—CH₂CH₂CH₂OMe, J=1H-pyrazol-1-ylmethyl.
53. Compound of Formula (I), wherein M=2,6-difluorophenyl, Q=4-tent-butyl-3-methoxyphenyl, Z=thiazol-2-ylmethyl, W═H, Y=—CH₂CH₂CH₂OMe, J=1H-pyrazol-1-ylmethyl.
54. Compound of Formula (I), wherein M=2-chlorophenyl, Q=4-tent-butyl-3-methoxyphenyl, Z=thiazol-2-ylmethyl, W═H, Y=—CH₂CH₂CH₂OMe, J=1H-pyrazol-1-ylmethyl.
55. Compound of Formula (I), wherein M=2-cyanophenyl, Q=4-tent-butyl-3-methoxyphenyl, Z=thiazol-2-ylmethyl, W═H, Y=—CH₂CH₂CH₂OMe, J=1H-pyrazol-1-ylmethyl.
56. Compound of Formula (I), wherein M=2-trifluoromethylphenyl, Q=4-tent-butyl-3-methoxyphenyl, Z=thiazol-2-ylmethyl, W═H, Y=—CH₂CH₂CH₂OMe, J=1H-pyrazol-1-ylmethyl.
57. Compound of Formula (I), wherein M=2-trifluoromethoxyphenyl, Q=4-tert-butyl-3-methoxyphenyl, Z=thiazol-2-ylmethyl, W═H, Y=—CH₂CH₂CH₂OMe, J=1H-pyrazol-1-ylmethyl.
58. Compound of Formula (I), wherein M=2-fluorophenyl, Q=4-tent-butyl-2-fluoro-3-methoxyphenyl, Z=thiazol-2-ylmethyl, W═H, Y=—CH₂CH₂CH₂OMe, J=1H-pyrazol-1-ylmethyl.
59. Compound of Formula (I), wherein M=2-fluorophenyl, Q=4-tert-butyl-2,6-difluoro-3-methoxyphenyl, Z=thiazol-2-ylmethyl, W═H, Y=—CH₂CH₂CH₂OMe, J=1H-pyrazol-1-ylmethyl.
60. Compound of Formula (I), wherein M=methyl, Q=4-tent-butyl-3-methoxyphenyl, Z=pyridin-2-ylmethyl, W═H, Y=—CH₂CH₂CN, J=1H-pyrazol-1-ylmethyl.
61. Compound of Formula (I), wherein M=methyl, Q=4-tent-butyl-3-methoxyphenyl, Z=(5-methylisoxazol-3-yl)methyl, W═H, Y=—CH₂CH₂CN, J=1H-pyrazol-1-ylmethyl.
62. Compound of Formula (Ia), wherein M=2-fluorophenyl, Q=5-bromo-4-tert-butyl-2-fluorophenyl, Z=pyridin-2-ylmethyl, W═H, Y=—CH₂CH₂CH₂OMe, J=1H-pyrazol-1-ylmethyl.
63. Compound of Formula (I), wherein M=Me, Q=3-bromo-4-tert-butylphenyl, Z=benzyl, W═H, Y=—CH₂CH₂CH₂OMe, J=1H-pyrazol-1-ylmethyl.
64. Compound of Formula (I), wherein M=2-thiophenyl, Q=4-tent-butyl-3-methoxyphenyl, Z=thiazol-2-ylmethyl, W═H, Y=—CH₂CH₂CH₂OMe, J=1H-pyrazol-1-ylmethyl.
65. Compound of Formula (I), wherein M=phenyl, Q=3-bromo-4-tert-butylphenyl, Z=thiazol-2-ylmethyl, W═H, Y=—CH₂CH₂CN, J=1H-pyrazol-1-ylmethyl.
66. Compound of Formula (I), wherein M=phenyl, Q=naphthalen-2-yl, Z=thiazol-2-ylmethyl, W═H, Y=—CH₂CH₂CN, J=1H-pyrazol-1-ylmethyl.
67. Compound of Formula (I), wherein M=phenyl, Q=4-tert-butyl-3-difluoromethylphenyl, Z=thiazol-2-ylmethyl, W═H, Y=—CH₂CH₂CN, J=1H-pyrazol-1-ylmethyl.
68. Compound of Formula (I), wherein M=phenyl, Q=4-tert-butyl-3-(1,1-difluoroethyl)phenyl, Z=thiazol-2-ylmethyl, W═H, Y=—CH₂CH₂CN, J=1H-pyrazol-1-ylmethyl.
69. Compound of Formula (I), wherein M=phenyl, Q=4-tent-butyl-3-difluoromethoxyphenyl, Z=thiazol-2-ylmethyl, W═H, Y=—CH₂CH₂CN, J=1H-pyrazol-1-ylmethyl.
70. Compound of Formula (I), wherein M=phenyl, Q=6-bromo-4-tert-butyl-3-methoxyphenyl, Z=thiazol-2-ylmethyl, W═H, Y=—CH₂CH₂CN, J=1H-pyrazol-1-ylmethyl.
71. Compound of Formula (I), wherein M=2-fluorophenyl, Q=4-tent-butyl-3-fluorophenyl, Z=thiazol-2-ylmethyl, W═H, Y=—CH₂CH₂CH₂OMe, J=1H-pyrazol-1-ylmethyl.
72. Compound of Formula (I), wherein M=2-fluorophenyl, Q=4-tert-butyl-3-chlorophenyl, Z=thiazol-2-ylmethyl, W═H, Y=—CH₂CH₂CH₂OMe, J=1H-pyrazol-1-ylmethyl.
73. Compound of Formula (I), wherein M=2-fluorophenyl, Q=4-tert-butyl-6-fluoro-3-methoxyphenyl, Z=thiazol-2-ylmethyl, W═H, Y=—CH₂CH₂CH₂OMe, J=1H-pyrazol-1-ylmethyl.
74. Compound of Formula (I), wherein M=2-fluorophenyl, Q=7-tent-butyl-benzoxazol-4-yl, Z=thiazol-2-ylmethyl, W═H, Y=—CH₂CH₂CH₂OMe, J=1H-pyrazol-1-ylmethyl.
75. Compound of Formula (I), wherein M=2-fluorophenyl, Q=4-tert-butyl-3-ethoxylphenyl, Z=thiazol-2-ylmethyl, W═H, Y=—CH₂CH₂CH₂OMe, J=1H-pyrazol-1-ylmethyl.
76. Compound of Formula (Ia), wherein M=2-fluorophenyl, Q=4-tert-butyl-6-fluoro-3-methoxyphenyl, Z=thiazol-2-ylmethyl, W═H, Y=—CH₂CH₂CH₂OMe, J=1H-pyrazol-1-ylmethyl.
77. The Opposite Enantiomer of Compound of Formula (Ia), wherein M=2-fluorophenyl, Q=4-tent-butyl-6-fluoro-3-methoxyphenyl, Z=thiazol-2-ylmethyl, W═H, Y=—CH₂CH₂CH₂OMe, J=1H-pyrazol-1-ylmethyl.
78. Compound of Formula (Ia), wherein M=2-fluorophenyl, Q=3-bromo-4-tert-butyl-6-fluorophenyl, Z=thiazol-2-ylmethyl, W═H, Y=—CH₂CH₂CH₂OMe, J=1H-pyrazol-1-ylmethyl.
79. Compound of Formula (Ia), wherein M=2-fluorophenyl, Q=3-bromo-4-tert-butyl-2-fluorophenyl, Z=thiazol-2-ylmethyl, W═H, Y=—CH₂CH₂CH₂OMe, J=1H-pyrazol-1-ylmethyl.
80. Compound of Formula (Ia), wherein M=2-fluorophenyl, Q=3-bromo-4-tert-butylphenyl, Z=thiazol-2-ylmethyl, W═H, Y=—CH₂CH₂CH₂OMe, J=1H-pyrazol-1-ylmethyl.
81. Compound of Formula (Ia), wherein M=2-fluorophenyl, Q=4-tert-butyl-3-methoxyphenyl, Z=thiazol-2-ylmethyl, W═H, Y=—CH₂CH₂CH₂OMe, J=1H-pyrazol-1-ylmethyl.

A further embodiment of the present invention includes pharmaceutical compositions comprising any single compound delineated herein, or principle embodiment or embodiment described therein, or a pharmaceutically acceptable salt, ester, solvate, or prodrug thereof, with a pharmaceutically acceptable carrier or excipient.

Yet another embodiment of the present invention is a pharmaceutical composition comprising a combination of two or more compounds delineated herein, or a pharmaceutically acceptable salt, ester, solvate, or prodrug thereof, with a pharmaceutically acceptable carrier or excipient.

Yet a further embodiment of the present invention is a pharmaceutical composition comprising any single compound delineated herein in combination with one or more HCV compounds known in the art, or a pharmaceutically acceptable salt, ester, solvate, or prodrug thereof, with a pharmaceutically acceptable carrier or excipient.

It will be appreciated that reference herein to therapy and/or treatment includes, but is not limited to prevention, retardation, prophylaxis, therapy and cure of the disease. It will further be appreciated that references herein to treatment or prophylaxis of HCV infection includes treatment or prophylaxis of HCV-associated disease such as liver fibrosis, cirrhosis and hepatocellular carcinoma.

It will be further appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

It will be further appreciated that the compounds of the invention, or their pharmaceutically acceptable salts, stereoisomers, tautomers, prodrugs or salt of a prodrug thereof, inhibit HCV polymerase, an RNA dependent RNA polymerase, an enzyme essential for HCV viral replication. Compounds of the present invention can be administered as the sole active pharmaceutical agent, or used in combination with one or more agents to treat or prevent hepatitis C infections or the symptoms associated with HCV infection. Other agents to be administered in combination with a compound or combination of compounds of the invention include therapies for disease caused by HCV infection that suppresses HCV viral replication by direct or indirect mechanisms. These include agents such as host immune modulators (for example, interferon-alpha, pegylated interferon-alpha, interferon-beta, interferon-gamma, CpG oligonucleotides and the like), or antiviral compounds that inhibit host cellular functions such as inosine monophosphate dehydrogenase (for example, ribavirin and the like). Also included are cytokines that modulate immune function. Also included are vaccines comprising HCV antigens or antigen adjuvant combinations directed against HCV. Also included are agents that interact with host cellular components to block viral protein synthesis by inhibiting the internal ribosome entry site (IRES) initiated translation step of HCV viral replication or to block viral particle maturation and release with agents targeted toward the viroporin family of membrane proteins such as, for example, HCV P7 and the like. Other agents to be administered in combination with a compound of the present invention include any agent or combination of agents that inhibit the replication of HCV by targeting proteins of the viral genome involved in the viral replication. These agents include but are not limited to other inhibitors of HCV RNA dependent RNA polymerase such as, for example, nucleoside type polymerase inhibitors described in WO0190121 (A2), or U.S. Pat. No. 6,348,587B1 or WO0160315 or WO0132153 or non-nucleoside inhibitors such as, for example, benzimidazole polymerase inhibitors described in EP1 162196A1 or WO0204425.

Accordingly, one aspect of the invention is directed to a method for treating or preventing an infection caused by an RNA-containing virus comprising co-administering to a patient in need of such treatment one or more agents selected from the group consisting of a host immune modulator and a second antiviral agent, or a combination thereof, with a therapeutically effective amount of a compound or combination of compounds of the invention, or a pharmaceutically acceptable salt, stereoisomer, tautomer, prodrug, salt of a prodrug, or combination thereof. Examples of the host immune modulator are, but not limited to, interferon-alpha, pegylated-interferon-alpha, interferon-beta, interferon-gamma, a cytokine, a vaccine, and a vaccine comprising an antigen and an adjuvant, and said second antiviral agent inhibits replication of HCV either by inhibiting host cellular functions associated with viral replication or by targeting proteins of the viral genome.

Further aspect of the invention is directed to a method of treating or preventing infection caused by an RNA-containing virus comprising co-administering to a patient in need of such treatment an agent or combination of agents that treat or alleviate symptoms of HCV infection including cirrhosis and inflammation of the liver, with a therapeutically effective amount of a compound or combination of compounds of the invention, or a pharmaceutically acceptable salt, stereoisomer, tautomer, prodrug, salt of a prodrug, or combination thereof. Yet another aspect of the invention provides a method of treating or preventing infection caused by an RNA-containing virus comprising co-administering to a patient in need of such treatment one or more agents that treat patients for disease caused by hepatitis B (HBV) infection, with a therapeutically effective amount of a compound or a combination of compounds of the invention, or a pharmaceutically acceptable salt, stereoisomer, tautomer, prodrug, salt of a prodrug, or combination thereof. An agent that treats patients for disease caused by hepatitis B (HBV) infection may be for example, but not limited thereto, L-deoxythymidine, adefovir, lamivudine or tenfovir, or any combination thereof. Example of the RNA-containing virus includes, but not limited to, hepatitis C virus (HCV).

Another aspect of the invention provides a method of treating or preventing infection caused by an RNA-containing virus comprising co-administering to a patient in need of such treatment one or more agents that treat patients for disease caused by human immunodeficiency virus (HIV) infection, with a therapeutically effective amount of a compound or a combination of compounds of the invention, or a pharmaceutically acceptable salt, stereoisomer, tautomer, prodrug, salt of a prodrug, or combination thereof. The agent that treats patients for disease caused by human immunodeficiency virus (HIV) infection may include, but is not limited thereto, ritonavir, lopinavir, indinavir, nelfmavir, saquinavir, amprenavir, atazanavir, tipranavir, TMC-114, fosamprenavir, zidovudine, lamivudine, didanosine, stavudine, tenofovir, zalcitabine, abacavir, efavirenz, nevirapine, delavirdine, TMC-125, L-870812, S-1360, enfuvirtide (T-20) or T-1249, or any combination thereof. Example of the RNA-containing virus includes, but not limited to, hepatitis C virus (HCV). In addition, the present invention provides the use of a compound or a combination of compounds of the invention, or a therapeutically acceptable salt form, stereoisomer, or tautomer, prodrug, salt of a prodrug, or combination thereof, and one or more agents selected from the group consisting of a host immune modulator and a second antiviral agent, or a combination thereof, to prepare a medicament for the treatment of an infection caused by an RNA-containing virus in a patient, particularly hepatitis C virus. Examples of the host immune modulator are, but not limited to, interferon-alpha, pegylated-interferon-alpha, interferon-beta, interferon-gamma, a cytokine, a vaccine, and a vaccine comprising an antigen and an adjuvant, and said second antiviral agent inhibits replication of HCV either by inhibiting host cellular functions associated with viral replication or by targeting proteins of the viral genome.

When used in the above or other treatments, combination of compound or compounds of the invention, together with one or more agents as defined herein above, can be employed in pure form or, where such forms exist, in pharmaceutically acceptable salt form, prodrug, salt of a prodrug, or combination thereof. Alternatively, such combination of therapeutic agents can be administered as a pharmaceutical composition containing a therapeutically effective amount of the compound or combination of compounds of interest, or their pharmaceutically acceptable salt form, prodrugs, or salts of the prodrug, in combination with one or more agents as defined hereinabove, and a pharmaceutically acceptable carrier. Such pharmaceutical compositions can be used for inhibiting the replication of an RNA-containing virus, particularly Hepatitis C virus (HCV), by contacting said virus with said pharmaceutical composition. In addition, such compositions are useful for the treatment or prevention of an infection caused by an RNA-containing virus, particularly Hepatitis C virus (HCV).

Hence, further aspect of the invention is directed to a method of treating or preventing infection caused by an RNA-containing virus, particularly a hepatitis C virus (HCV), comprising administering to a patient in need of such treatment a pharmaceutical composition comprising a compound or combination of compounds of the invention or a pharmaceutically acceptable salt, stereoisomer, or tautomer, prodrug, salt of a prodrug, or combination thereof, one or more agents as defined hereinabove, and a pharmaceutically acceptable carrier.

When administered as a combination, the therapeutic agents can be formulated as separate compositions which are given at the same time or within a predetermined period of time, or the therapeutic agents can be given as a single unit dosage form.

Antiviral agents contemplated for use in such combination therapy include agents (compounds or biologicals) that are effective to inhibit the formation and/or replication of a virus in a mammal, including but not limited to agents that interfere with either host or viral mechanisms necessary for the formation and/or replication of a virus in a mammal. Such agents can be selected from another anti-HCV agent; an HIV inhibitor; an HAV inhibitor; and an HBV inhibitor.

Other anti-HCV agents include those agents that are effective for diminishing or preventing the progression of hepatitis C related symptoms or disease. Such agents include but are not limited to immunomodulatory agents, inhibitors of HCV NS3 protease, other inhibitors of HCV polymerase, inhibitors of another target in the HCV life cycle and other anti-HCV agents, including but not limited to ribavirin, amantadine, levovirin and viramidine.

Immunomodulatory agents include those agents (compounds or biologicals) that are effective to enhance or potentiate the immune system response in a mammal. Immunomodulatory agents include, but are not limited to, inosine monophosphate dehydrogenase inhibitors such as VX-497 (merimepodib, Vertex Pharmaceuticals), class I interferons, class II interferons, consensus interferons, asialo-interferons pegylated interferons and conjugated interferons, including but not limited to interferons conjugated with other proteins including but not limited to human albumin. Class I interferons are a group of interferons that all bind to receptor type I, including both naturally and synthetically produced class I interferons, while class II interferons all bind to receptor type II. Examples of class I interferons include, but are not limited to, [alpha]-, [beta]-, [delta]-, [omega]-, and [tau]-interferons, while examples of class II interferons include, but are not limited to, [gamma]-interferons.

Inhibitors of HCV NS3 protease include agents (compounds or biologicals) that are effective to inhibit the function of HCV NS3 protease in a mammal Inhibitors of HCV NS3 protease include, but are not limited to, those compounds described in WO 99/07733, WO 99/07734, WO 00/09558, WO 00/09543, WO 00/59929, WO 03/064416, WO 03/064455, WO 03/064456, WO 2004/030670, WO 2004/037855, WO 2004/039833, WO 2004/101602, WO 2004/101605, WO 2004/103996, WO 2005/028501, WO 2005/070955, WO 2006/000085, WO 2006/007700 and WO 2006/007708 (all by Boehringer Ingelheim), WO 02/060926, WO 03/053349, WO03/099274, WO 03/099316, WO 2004/032827, WO 2004/043339, WO 2004/094452, WO 2005/046712, WO 2005/051410, WO 2005/054430 (all by BMS), WO 2004/072243, WO 2004/093798, WO 2004/113365, WO 2005/010029 (all by Enanta), WO 2005/037214 (Intermune) and WO 2005/051980 (Schering), and the candidates identified as VX-950, ITMN-191 and SCH 503034.

Inhibitors of HCV polymerase include agents (compounds or biologicals) that are effective to inhibit the function of an HCV polymerase. Such inhibitors include, but are not limited to, non-nucleoside and nucleoside inhibitors of HCV NS5B polymerase. Examples of inhibitors of HCV polymerase include but are not limited to those compounds described in: WO 02/04425, WO 03/007945, WO 03/010140, WO 03/010141, WO 2004/064925, WO 2004/065367, WO 2005/080388 and WO 2006/007693 (all by Boehringer Ingelheim), WO 2005/049622 (Japan Tobacco), WO 2005/014543 (Japan Tobacco), WO 2005/012288 (Genelabs), WO 2004/087714 (IRBM), WO 03/101993 (Neogenesis), WO 03/026587 (BMS), WO 03/000254 (Japan Tobacco), and WO 01/47883 (Japan Tobacco), and the clinical candidates XTL-2125, HCV 796, R-1626 and NM 283.

Inhibitors of another target in the HCV life cycle include agents (compounds or biologicals) that are effective to inhibit the formation and/or replication of HCV other than by inhibiting the function of the HCV NS3 protease. Such agents may interfere with either host or HCV viral mechanisms necessary for the formation and/or replication of HCV. Inhibitors of another target in the HCV life cycle include, but are not limited to, entry inhibitors, agents that inhibit a target selected from a helicase, a NS2/3 protease and an internal ribosome entry site (IRES) and agents that interfere with the function of other viral targets including but not limited to an NS5A protein and an NS4B protein.

It can occur that a patient may be co-infected with hepatitis C virus and one or more other viruses, including but not limited to human immunodeficiency virus (HIV), hepatitis A virus (HAV) and hepatitis B virus (HBV). Thus also contemplated is combination therapy to treat such co-infections by co-administering a compound according to the present invention with at least one of an HIV inhibitor, an HAV inhibitor and an HBV inhibitor.

Other agents to be administered in combination with a compound of the present invention include a cytochrome P450 monooxygenase inhibitor (also referred to herein as a CYP inhibitor), which is expected to inhibit metabolism of the compounds of the invention. Therefore, the cytochrome P450 monooxygenase inhibitor would be in an amount effective to inhibit metabolism of the compounds of this invention. Accordingly, the CYP inhibitor is administered in an amount such that the bioavailiablity of the protease inhibitor is increased in comparison to the bioavailability in the absence of the CYP inhibitor.

In one embodiment, the invention provides methods for improving the pharmacokinetics of compounds of the invention. The advantages of improving the pharmacokinetics of drugs are recognized in the art (see, for example, US Patent App. Nos. 2004/0091527; US 2004/0152625; and US 2004/0091527). Accordingly, one embodiment of this invention provides a method for administering an inhibitor of CYP3A4 and a compound of the invention. Another embodiment of this invention provides a method for administering a compound of the invention and an inhibitor of isozyme 3A4 (“CYP3A4”), isozyme 2C19 (“CYP2C19”), isozyme 2D6 (“CYP2D6”), isozyme 1A2 (“CYP1A2”), isozyme 2C9 (“CYP2C9”), or isozyme 2E1 (“CYP2E1”). In a preferred embodiment, the CYP inhibitor preferably inhibits CYP3A4. Any CYP inhibitor that improves the pharmacokinetics of the relevant NS3/4A protease may be used in a method of this invention. These CYP inhibitors include, but are not limited to, ritonavir (see, for example, WO 94/14436), ketoconazole, troleandomycin, 4-methyl pyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944, and VX-497. Preferred CYP inhibitors include ritonavir, ketoconazole, troleandomycin, 4-methyl pyrazole, cyclosporin, and clomethiazole.

It will be understood that the administration of the combination of the invention by means of a single patient pack, or patient packs of each formulation, containing within a package insert instructing the patient to the correct use of the invention is a desirable additional feature of this invention.

According to a further aspect of the invention is a pack comprising at least a compound of the invention and a CYP inhibitor of the invention and an information insert containing directions on the use of the combination of the invention. In an alternative embodiment of this invention, the pharmaceutical pack further comprises one or more of additional agent as described herein. The additional agent or agents may be provided in the same pack or in separate packs.

Another aspect of this invention involves a packaged kit for a patient to use in the treatment of HCV infection or in the prevention of HCV infection, comprising: a single or a plurality of pharmaceutical formulation of each pharmaceutical component; a container housing the pharmaceutical formulation (s) during storage and prior to administration; and instructions for carrying out drug administration in a manner effective to treat or prevent HCV infection.

Accordingly, this invention provides kits for the simultaneous or sequential administration of a compound of the invention and a CYP inhibitor (and optionally an additional agent) or derivatives thereof are prepared in a conventional manner. Typically, such a kit will comprise, e.g. a composition of each inhibitor and optionally the additional agent (s) in a pharmaceutically acceptable carrier (and in one or in a plurality of pharmaceutical formulations) and written instructions for the simultaneous or sequential administration.

In another embodiment, a packaged kit is provided that contains one or more dosage forms for self administration; a container means, preferably sealed, for housing the dosage forms during storage and prior to use; and instructions for a patient to carry out drug administration. The instructions will typically be written instructions on a package insert, a label, and/or on other components of the kit, and the dosage form or forms are as described herein. Each dosage form may be individually housed, as in a sheet of a metal foil-plastic laminate with each dosage form isolated from the others in individual cells or bubbles, or the dosage forms may be housed in a single container, as in a plastic bottle. The present kits will also typically include means for packaging the individual kit components, i.e., the dosage forms, the container means, and the written instructions for use. Such packaging means may take the form of a cardboard or paper box, a plastic or foil pouch, etc.

### DEFINITIONS

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system including, but not limited to, phenyl, naphthyl, tetrahydronaphthyl, indanyl, idenyl.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl.

In accordance with the invention, any of the aryls, substituted aryls, heteroaryls and substituted heteroaryls described herein, can be any aromatic group. Aromatic groups can be substituted or unsubstituted.

The terms “C₁-C₄ alkyl,” “C₅-C₈ alkyl,” or “C₁-C₈ alkyl,” as used herein, refer to saturated, straight- or branched-chain hydrocarbon radicals containing between one and four, five and eight, or one and eight carbon atoms, respectively. Examples of C₁-C₈ alkyl radicals include, but are not limited to, methyl, ethyl, propyl, isopropyl, n-butyl, tent-butyl, neopentyl, n-hexyl, heptyl and octyl radicals.

The terms “C₂-C₈ alkenyl,” or “C₃-C₆ alkenyl,” as used herein, refer to straight- or branched-chain hydrocarbon radicals containing from two to eight, or three and six carbon atoms having at least one carbon-carbon double bond by the removal of a single hydrogen atom. Alkenyl groups include, but are not limited to, for example, ethenyl, propenyl, butenyl, 1-methyl-2-buten-1-yl, heptenyl, octenyl, and the like.

The terms “C₂-C₈ alkynyl,” or “C₃-C₆ alkynyl,” as used herein, refer to straight- or branched-chain hydrocarbon radicals containing from two to eight, or three and six carbon atoms having at least one carbon-carbon triple bond by the removal of a single hydrogen atom. Representative alkynyl groups include, but are not limited to, for example, ethynyl, 1-propynyl, 1-butynyl, heptynyl, octynyl, and the like.

The term “C₃-C₈-cycloalkyl”, or “C₃-C₁₂-cycloalkyl,” as used herein, refers to a monocyclic or polycyclic saturated carbocyclic ring compound. Examples of C₃-C₈-cycloalkyl include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo [2.2.1] heptyl, and bicyclo [2.2.2] octyl.

The term “C₃-C₈ cycloalkenyl”, or “C₃-C₁₂ cycloalkenyl” as used herein, refers to monocyclic or polycyclic carbocyclic ring compound having at least one carbon-carbon double bond. Examples of C₃-C₈ cycloalkenyl include, but not limited to, cyclopropenyl, cyclobutenyl, cyclopentenyl, cyclohexenyl, cycloheptenyl, cyclooctenyl, and the like; and examples of C₃-C₁₂ cycloalkenyl include, but not limited to, cyclopropenyl, cyclobutenyl, cyclopentenyl, cyclohexenyl, cycloheptenyl, cyclooctenyl, and the like.

It is understood that any alkyl, alkenyl, alkynyl and cycloalkyl moiety described herein can also be an aliphatic group, an alicyclic group or a heterocyclic group. An “aliphatic” group is a non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

The term “alicyclic,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo [2.2.1] heptyl, and bicyclo [2.2.2] octyl. Such alicyclic groups may be further substituted.

The terms “heterocyclic” or “heterocycloalkyl” can be used interchangeably and referred to a non-aromatic ring or a bi- or tri-cyclic group fused system, where (i) each ring system contains at least one heteroatom independently selected from oxygen, sulfur and nitrogen, (ii) each ring system can be saturated or unsaturated, (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl. Such heterocyclic groups may be further substituted.

The term “substituted” refers to substitution by independent replacement of one, two, or three or more of the hydrogen atoms thereon with substituents including, but not limited to, —F, —Cl, —Br, —I, —OH, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₈-alkenyl, —SO₂NH—C₂-C₈-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthiomethyl. It is understood that the aryls, heteroaryls, alkyls, and the like can be further substituted.

The term “halogen,” as used herein, refers to an atom selected from fluorine, chlorine, bromine and iodine.

The term “hydrogen” includes hydrogen and deuterium. In addition, the recitation of an atom includes other isotopes of that atom so long as the resulting compound is pharmaceutically acceptable.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including mesylate, tosylate, triflate, p-nitrobenzoate, phosphonate groups, for example.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in T. H. Greene and P. G. M. Wuts, Protective Groups in Organic Synthesis, 3rd edition, John Wiley & Sons, New York (1999). Examples of hydroxyl protecting groups include benzyloxycarbonyl, 4-methoxybenzyloxycarbonyl, tert-butoxycarbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenylmethyl (trityl), methoxymethyl, methylthiomethyl, benzyloxymethyl, 2-(trimethylsilyl)ethoxymethyl, methanesulfonyl, trimethylsilyl, triisopropylsilyl, and the like.

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “protic solvent” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject” as used herein refers to an animal. Preferably the animal is a mammal. More preferably the mammal is a human. A subject also refers to, for example, dogs, cats, horses, cows, pigs, guinea pigs, fish, birds and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs,” as used herein, refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug” as used herein means a compound which is convertible in vivo by metabolic means (e.g. by hydrolysis) to a compound of the invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

The present invention also relates to solvates of the compounds of Formula (I), for example hydrates.

This invention also encompasses pharmaceutical compositions containing, and methods of treating viral infections through administering, pharmaceutically acceptable prodrugs of compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminun hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43650 by Montgomery, all of which are incorporated herein by reference). A discussion of pulmonary delivery of antibiotics is also found in U.S. Pat. No. 6,014,969, incorporated herein by reference.

### Antiviral Activity

An inhibitory amount or dose of the compounds of the present invention may range from about 0.01 mg/Kg to about 500 mg/Kg, alternatively from about 1 to about 50 mg/Kg. Inhibitory amounts or doses will also vary depending on route of administration, as well as the possibility of co-usage with other agents.

According to the methods of treatment of the present invention, viral infections, conditions are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the present invention described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically exipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of the Formula described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The said “additional therapeutic or prophylactic agents” includes but not limited to, immune therapies (eg. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (eg N-acetylcysteine), cytokine agonists, cytokine antagonists, lung surfactants and/or antimicrobial and anti-viral agents (eg ribavirin and amantidine). The compositions according to the invention may also be used in combination with gene replacement therapy.

Unless otherwise defined, all technical and scientific terms used herein are accorded the meaning commonly known to one of ordinary skill in the art. All publications, patents, published patent applications, and other references mentioned herein are hereby incorporated by reference in their entirety.

### Abbreviations

Abbreviations which may be used in the descriptions of the scheme and the examples that follow are: Ac for acetyl; AcOH for acetic acid; AIBN for azobisisobutyronitrile; BINAP for 2,2′-bis(diphenylphosphino)-1,1′-binaphthyl; Boc₂O for di-tert-butyl-dicarbonate; Boc for t-butoxycarbonyl; Bpoc for 1-methyl-1-(4-biphenylyl)ethyl carbonyl; Bz for benzoyl; Bn for benzyl; BocNHOH for tent-butyl N-hydroxycarbamate; t-BuOK for potassium tert-butoxide; Bu₃SnH for tributyltin hydride; BOP for (benzotriazol-1-yloxy)tris(dimethylamino)phosphonium Hexafluorophosphate; Brine for sodium chloride solution in water; CDI for carbonyldiimidazole; CH₂Cl₂ for dichloromethane; CH₃ for methyl; CH₃CN for acetonitrile; Cs₂CO₃ for cesium carbonate; CuCl for copper (I) chloride; CuI for copper (I) iodide; dba for dibenzylidene acetone; dppb for diphenylphosphino butane; DBU for 1,8-diazabicyclo[5.4.0]undec-7-ene; DCC for N,N′-dicyclohexylcarbodiimide; DEAD for diethylazodicarboxylate; DIAD for diisopropyl azodicarboxylate; DIPEA or (i-Pr)₂EtN for N,N,-diisopropylethyl amine; Dess-Martin periodinane for 1,1,1-tris(acetyloxy)-1,1-dihydro-1,2-benziodoxol-3-(1H)-one; DMAP for 4-dimethylaminopyridine; DME for 1,2-dimethoxyethane; DMF for N,N-dimethylformamide; DMSO for dimethyl sulfoxide; DMT for di(p-methoxyphenyl)phenylmethyl or dimethoxytrityl; DPPA for diphenylphosphoryl azide; EDC for N-(3-dimethylaminopropyl)-N′-ethylcarbodiimide; EDC HCl for N-(3-dimethylaminopropyl)-N′-ethylcarbodiimide hydrochloride; EtOAc for ethyl acetate; EtOH for ethanol; Et₂O for diethyl ether; HATU for O-(7-azabenzotriazol-1-yl)-N,N,N′,N′,-tetramethyluronium Hexafluorophosphate; HCl for hydrogen chloride; HOBT for 1-hydroxybenzotriazole; K₂CO₃ for potassium carbonate; n-BuLi for n-butyl lithium; i-BuLi for i-butyl lithium; t-BuLi for t-butyl lithium; PhLi for phenyl lithium; LDA for lithium diisopropylamide; LiTMP for lithium 2,2,6,6-tetramethylpiperidinate; MeOH for methanol; Mg for magnesium; MOM for methoxymethyl; Ms for mesyl or —SO₂—CH₃; Ms₂O for methanesulfonic anhydride or mesyl-anhydride; NaBH₄ for sodium borohydride; NaBH₃CN for sodium cyanoborohydride; NaN(TMS)₂ for sodium bis(trimethylsilyl)amide; NaCl for sodium chloride; NaH for sodium hydride; NaHCO₃ for sodium bicarbonate or sodium hydrogen carbonate; Na₂CO₃ sodium carbonate; NaOH for sodium hydroxide; Na₂SO₄ for sodium sulfate; NaHSO₃ for sodium bisulfite or sodium hydrogen sulfite; Na₂S₂O₃ for sodium thiosulfate; NH₂NH₂ for hydrazine; NH₄HCO₃ for ammonium bicarbonate; NH₄Cl for ammonium chloride; NMMO for N-methylmorpholine N-oxide; NaIO₄ for sodium periodate; Ni for nickel; OH for hydroxyl; OsO₄ for osmium tetroxide; TBAF for tetrabutylammonium fluoride; TEA or Et₃N for triethylamine; TFA for trifluoroacetic acid; THF for tetrahydrofuran; TMEDA for N,N,N′,N′-tetramethylethylenediamine; TPP or PPh₃ for triphenyl-phosphine; Troc for 2,2,2-trichloroethyl carbonyl; Ts for tosyl or SO₂—C₆H₄—CH₃; Ts₂O for tolylsulfonic anhydride or tosyl-anhydride; TsOH for p-tolylsulfonic acid; Pd for palladium; Ph for phenyl; POPd for dihydrogen dichlorobis(di-tert-butylphosphinito-κP)palladate(II); Pd₂(dba)₃ for tris(dibenzylideneacetone) dipalladium (0); Pd(PPh₃)₄ for tetrakis(triphenylphosphine)palladium (0); PdCl₂(PPh₃)₂ for trans-dichlorobis-(triphenylphosphine)palladium (II); Pt for platinum; Rh for rhodium; Ru for ruthenium; TBS for tent-butyl dimethylsilyl; TMS for trimethylsilyl; or TMSCl for trimethylsilyl chloride.

### Synthetic Methods

The compounds and processes of the present invention will be better understood in connection with the following synthetic schemes that illustrate the methods by which the compounds of the invention may be prepared.

The compounds of the present invention may be prepared via several different synthetic routes. The most straightforward method is shown in Schemes 1 and 2, in which M, W, Q, Z, Y and J are as previously defined and LG is a leaving group such as but not limited to chloride, Ms, benzotriazolyl, hydroxyl, or the like; and PG is a carboxylic acid hydroxy protecting group, for example benzyl, tert-butyl, methyl or the like. Scheme 2 illustrates the syntheses of various acyl sulfonamide derivatives (I) from the corresponding carboxylic acids (2-1) or their protected forms (1-6), which can be synthesized using the procedures described in scheme 1.

The synthesis of the common intermediate (1-6) can be started from an imine intermediate (1-2) through alkyklation or Michael addition conditions. Imine (1-2) can be obtained by condensation of a α-amino carbonyl species, typically an amino acid derivative such as tert-butyl 2-amino-3-(1,3-thiazol-4-yl)-propanoate, tert-butyl 3-(1H-pyrazol-1-yl)-propanoate, benzyl 2-amino-3-(tent-butyldimethylsilyloxy)-propanoate, 2-amino-4-methyl-pentanoate, or the like, with an aldehyde (1-1.1, wherein Ar is an optionally substituted aryl or heteroaryl) promoted by a water-scavenger such as but not limited to magnesium sulfate, molecular sieves, methyl orthoformate, or the like; optionally in the presence of an acid such as but not limited to acetic acid, p-toluenesulfonic acid, lithium bromide, or the like, in an aprotic solvent at a temperature typically between −20° C. and 100° C. The preferred temperature is 0° C. to room temperature. Imine (1-2) can be deprotonated by a base such as but not limited to NaH, LDA, t-BuLi, PhLi, LiTMP, triethylamine, DBU, pyridine, K₂CO₃, NaHCO₃, lithium tert-butoxide, or the like, optionally in the presence of a phase-transfer catalyst such as tetrabutylammonium iodide, benzyl triethylammonium chloride, 18-crown-6 or the like; or a combination of a Lewis acid and a suitable base such as but not limited to lithium bromide and triethylamine; and the resulting reactive carbon anion can be trapped by a suitable electrophile which is known to those in the art, such as an aldehyde, Michael acceptor, or reagent (1-2.1), optionally in the presence of a palladium catalyst such as Pd₂(dba)₃, Pd(PPh₃)₄, PdCl₂(PPh₃)₂, or the like, and optionally in the presence of a ligand including but are not limited to PPh₃, AsPh₃, trimethyl phosphite, dppb, tri-o-tolyl-phosphine, or the like; in an aprotic solvent at a temperature typically between 20° C. and 100° C. (1-2.1) is a reactive species, selected from a group such as but not limited to 3-methoxypropyl iodide, benzyl chloride, allyl bromide, propargyl bromide, allyl acetate, allyl tert-butyl carbonate, methoxymethyl bromide, ethyl 3-bromoacrylate, or the like. The amino capping group in imine (1-3) can be removed to give amine (1-4) by hydrolyzing with water; optionally in the presence of a base such as NaHCO₃, NaOH, or the like, or an acid such as but not limited to citric acid, acetic acid, hydrochloric acid; at a temperature typically between 0° C. and 100° C. The amine (1-4) can be mono-substituted to (1-5) with reagent (1-4.1), such as but not limited to 2-pyridylmethyl bromide, benzyl chloride, 2-phenylethyl iodide, or the like, optionally in the presence of a base such as NaHCO₃, K₂CO₃, pyridine, triethylamine, n-butyllithium, or the like in an aprotic solvent at a temperature between −78° C. and 180° C. Alternatively the conversion from (1-4) to (1-5) can be realized using the well-known procedure of reductive amination conditions, with a suitable substituted carbonyl moiety such as 2-formylpyridine, acetophenone or the like. Yet alternatively (1-5) can also be obtained directly from the reductive amination of the suitable substituted imine (1-2). The secondary amine (1-5) is converted to a compound of formula (I-6) by reaction with reagent (1-5.1) in the presence of a base such as but not limited to triethylamine, pyridine, K₂CO₃, or the like, optionally in the presence of a condensation reagent which is known in the art such as EDC, HATU, or the like, in an aprotic solvent at a temperature typically between 0° C. and 100° C., preferably at room temperature. Deprotection of (1-6) will afford the carboxylic acid (2-1) under the conditions which are known to those in the art.

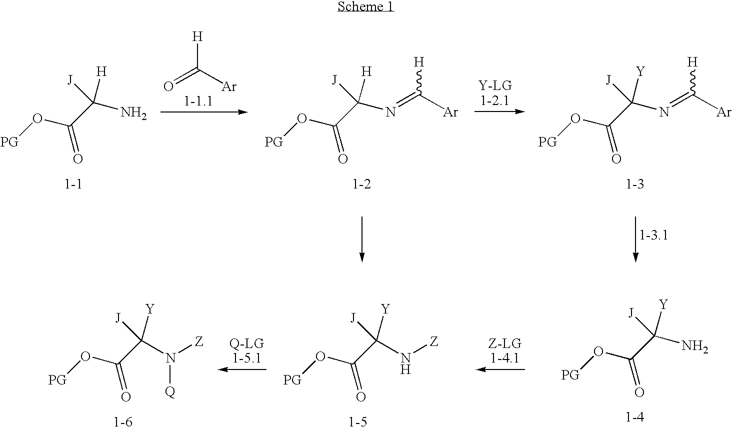

The compounds of the present invention may be derived from the carboxylic acid (2-1) as a common intermediate, through functional group manipulation which is well known to those in the art. For example, acid (2-1) can be activated to compound (2-2), wherein AG is an acid activating group such as imidazole, imidazolylcarboxy, chloride, methoxycarboxy, t-butylcarboxy, BOP-O—, EDC-O—, benzotriazol-1-yl-O— or the like, by reacting with CDI, thionyl or oxallyl chloride optionally in the presence of DMF, methyl chloroformate, t-butylcarbonyl chloride, BOP-Cl, EDC or EDCI, benzotriazol-1-ol in the presence of DCC, or the like, optionally in the presence of a base such as NaHCO₃, K₂CO₃, pyridine, Et₃N, DMAP, DBU or the like, in an aprotic solvent such as CH₂Cl₂, DMF, CH₃CN, toluene, pyridine, THF, or the like, at temperature from 0° C. to 100° C. preferably at room temperature. An amino-containing species (M-S(O)₂—N(W)H) then reacts with (2-2) in the presence of a base such as NaHCO₃, K₂CO₃, pyridine, Et₃N, DMAP, n-BuLi, DBU or the like, in an aprotic solvent such as CH₂Cl₂, DMF, CH₃CN, toluene, pyridine, THF, or the like, at temperature from 0° C. to 100° C. preferably at room temperature, to give the title compounds (I). Alternatively in certain cases, the above two-step process can be combined into a one-pot reaction. Alternatively the synthese of the title compounds (1) can be realized from the direct condensation of acid (2-1) with the amino species (M-S(O)₂—N(W)H) under various conditions which are known to those in the art.

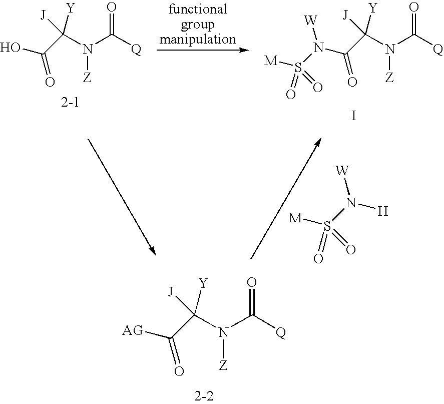

It will be appreciated that compounds of Formula (I), (1-4), (1-5), (1-6) and/or (2-1) which exist as diastereoisomers may optionally be separated by techniques well known in the art, for example by column chromatography.

It will be appreciated that racemic compounds of Formula (I), (1-4), (1-5), (1-6) and/or (2-1) may be optionally resolved into their individual enantiomers. Such resolutions may conveniently be accomplished by standard methods known in the art. For example, a racemic compound of Formula (I), (I-4), (1-5), (1-6) and/or (2-1) may be resolved by chiral preparative HPLC. Alternatively, racemic compounds of Formula (I), (1-4), (1-5), (1-6) and/or (2-1) which contain an appropriate acidic or basic group, such as a carboxylic acid group or amine group may be resolved by standard diastereoisomeric salt formation with a chiral base or acid reagent respectively as appropriate. Such techniques are well established in the art. For example, a racemic compound of Formula (1-4) or (1-5) may be resolved by treatment with a chiral acid such as (R)-(−)-1,1′-binaphthyl-2,2′-diyl-hydrogen phosphate, in a suitable solvent, for example dichloromethane, isopropanol or acetonitrile. The enantiomer of Formula (1-4) or (1-5) may then be obtained by treating the salt with a suitable base, for example triethylamine, in a suitable solvent, for example methyl tert-butyl ether. Individual enantiomers of Formula (1-4) and/or (1-5) may then be progressed to an enantiomeric compound of Formula (I) by the chemistry described above in respect of racemic compounds.

It will also be appreciated that individual enantiomeric compounds of Formula (1-4) and/or (1-5) may be prepared by general methods of asymmetric synthesis using, where appropriate, chiral auxiliaries or chiral catalytic reagents and additionally performing any suitable functional group interconversion step as hereinbefore described, including the addition or removal of any such chiral auxiliary. Such general methods of asymmetric synthesis are well known in the art and include, but are not restricted to, those described in “Asymmetric Synthesis,” Academic Press, 1984 and/or “Chiral Auxiliaries and Ligands in Asymmetric Synthesis,” Wiley, 1995. For example, suitable general chiral auxiliaries include chiral alcohols such as menthol or 1-phenylethanol; chiral oxazolidinones such as 4-benzyloxazolidin-2-one or 4-isopropyloxazolidin-2-one; chiral sultams such as camphor sultam; or chiral amines such as 1-phenylethylamine or 2-amino-2-phenylethanol. Suitable general chiral catalytic reagents include chiral basic amines and chiral ligands such as N-methylephedrine, 1-phenyl-2-(1-pyrrolidinyl)-1-propanol, 3-(dimethylamino)-1,7,7-trimethylbicyclo[2.2.1]-heptan-2-ol, 3,4-bis(diphenylphosphanyl)-1-(phenylmethyl)-pyrrolidine, chinchonine, chinchonidine, sparteine, hydroquinine or quinine, BINAP or chiral bis(oxazoline) (BOX) ligands and derivatives, optionally in the presence of a metal salt, for example AₐB_{b} where A is silver, cobalt, zinc, titanium, magnesium, or manganese, and B is halide (for example chloride or bromide), acetate, trifluoroacetate, p-toluenesulfonate, trifluoromethylsulfonate, hexafluorophosphate or nitrate, and ₐ, and _{b}, are 1, 2, 3 or 4, and optionally in the presence of a base, for example triethylamine. All of these chiral auxiliaries or chiral catalytic reagents are well described in the art. General illustrative examples of the preparation of various chiral pyrrolidines by asymmetric synthesis using chiral auxiliaries or chiral catalytic reagents include, but are not limited to, those described in Angew. Chem. Int. Ed., (2002), 41, 4236; Chem. Rev., (1998), 98, 863; J. Am. Chem. Soc., (2002), 124, 13400; J. Am. Chem. Soc., (2003), 125, 10175; Org. Lett., (2003), 5, 5043; Tetrahedron, (1995), 51, 273; Tetrahedron: Asymm., (1995), 6, 2475; Tetrahedron: Asymm., (2001), 12, 1977; Tetrahedron: Asymm., (2002), 13, 2099 and Tet. Lett., (1991), 41, 5817. It will be appreciated that, with appropriate manipulation and protection of any chemical functionality, synthesis of compounds of Formula (I) is accomplished by methods analogous to those above and to those described in the Experimental section. Suitable protecting groups can be found, but are not restricted to, those found in T W Greene and P G M Wuts “Protective Groups in Organic Synthesis”, 3rd Ed (1999), J Wiley and Sons.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

### EXAMPLES

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Although the invention has been described with respect to various preferred embodiments, it is not intended to be limited thereto, but rather those skilled in the art will recognize that variations and modifications may be made therein which are within the spirit of the invention and the scope of the appended claims.

### Example 1

### Compound of Formula (I), wherein M=Me, Q=4-tent-butyl-3-methoxyphenyl, Z=thiazol-2-ylmethyl, W═H, Y=—CH₂CH═CH₂, J=1H-pyrazol-1-ylmethyl

Step 1a. Into a suspension of commercially available 1-carboxy-2-pyrazol-1-yl-ammonium chloride (958 mg, 1.0 mmol) in t-butyl acetate (30.0 mL) was added perchloric acid (70%, 0.50 mL, 5.8 mmol). The mixture was stirred at room temperature for 64 hours before being diluted with ethyl acetate and neutralized with a combination of solid NaHCO₃ and saturated NaHCO₃ until no gas evolved. After separation, the aqueous was saturated with sodium chloride and extracted with ethyl acetate. The combined organics were dried (Na₂SO₄) and evaporated to give the crude product (617 mg, 45.5%). ESIMS m/z=212.12 [M+H]⁺ of the free base parent ion. ¹³C NMR (CDCl₃) 175.7, 171.1, 140.1, 130.5, 105.6, 82.6, 55.1, 54.2, 27.9.

Step 1b. Into a suspension of commercially available 1-carboxy-2-pyrazol-1-yl-ammonium chloride (958 mg, 1.0 mmol) in t-butyl acetate (30.0 mL) was added perchloric acid (70%, 0.76 mL, 8.8 mmol). The mixture was stirred at room temperature for 22 hours before being diluted with ethyl acetate and neutralized with a combination of solid NaHCO₃ and saturated NaHCO₃ to pH ˜8. After separation, the aqueous was saturated with sodium chloride and extracted with ethyl acetate. The combined organics were dried (Na₂SO₄) and evaporated to give the crude product (633 mg, 60%). ESIMS m/z=212.14 [M+H]⁺.

Step 1c. A mixture of the compound from step 1a (205 mg, 0.75 mmol), commercially available 2-formyl-1,3-thiazole (120 mg, 1.06 mmol), and activated molecular sieves (4 Å, 1.0 g) in anhydrous methylene chloride (5 mL) was stirred at room temperature for 15 hours before being filtered through Celite and washed with methylene chloride. The combined organics were evaporated and the residue was used directly for next step. ESIMS m/z=307.13 [M+H]⁺.

Step 1d. A mixture of the compound from step 1b (160 mg, 0.76 mmol), commercially available 2-formyl-1,3-thiazole (151 mg, 1.34 mmol), and activated molecular sieves (4 Å, 1.0 g) in anhydrous methylene chloride (5 mL) was stirred at room temperature for 15 hours before being filtered through Celite and washed with methylene chloride. The combined organics were evaporated and the residue was chromatographed (silica, hexanes-EtOAc) to give the desired compound (200 mg, 86%). ESIMS m/z=307.12 [M+H]⁺. ¹³C NMR (CD₃OD) 168.2, 166.2, 159.0, 144.1, 139.8, 131.4, 123.3, 105.4, 82.7, 72.3, 53.1, 27.1.

Step 1e. A mixture of the compound from step 1d (14.5 g, 47.4 mmol), allylbromide (12.2 ml, 142 mmol) and tetra-n-butylammonium iodide (17.6 g, 47.4 mmol) in 100 mL of toluene was treated with KOH (15.9 g, 284 mmol) at room temperature for 10 minutes before being filtered through a celite pad. The filtrate was washed (brine), dried (Na₂SO₄) and evaporated. The residue was used directly at the next step.

Into a solution of the above residue in MeCN (100 mL) containing bromocresol green (2 mg) was charged NaBH₃CN (6 g, 94.8 mmol). Acetic acid was added dropwise until the solution turned to yellow. The reaction was then diluted with water and Et₂O. The aqueous phase was extracted (EtOAc). The organics were washed (brine), dried (Na₂SO₄), and evaporated. The residue was chromatographed (silica, hexanes-EtOAc) to give the desired compound (3.2 g, 19.3%) as a light yellow oil. ESIMS m/z=349.09 [M+H]⁺. ¹³C NMR (CDCl₃) 172.5, 172.1, 142.7, 139.7, 132.2, 131.0, 119.7, 119.1, 105.8, 82.7, 65.9, 55.5, 45.3, 38.3, 28.2.

Step 1f. A mixture of the commercially available 4-t-butyl-3-methoxybenzoic acid (2.082 g, 10.0 mmol) in thionyl chloride (5.0 mL) was refluxed for 2.5 hours before being evaporated. Toluene (twice) was added to the residue and the mixture was evaporated. The residue was dried in vacuum to get the desired compound as a crystalline (2.258 g, 99.6%).

Step 1g. A solution of the compound from step 1e (120 mg, 0.34 mmol) in CH₂Cl₂ (1.0 mL) was treated with Et₃N (0.1 mL) and the compound of step 1f (130 mg, 0.51 mmol) at room temperature for 5 days before partition (EtOAc-water). The organics were washed (NaHCO₃, water, brine), dried (Na₂SO₄), and evaporated. The residue was chromatographed (silica, hexanes-EtOAc) to give the title compound (82 mg, 52%) as light yellow amorphous solid. ESIMS m/z=539.08 [M+H]⁺. ¹³C NMR (CDCl₃) 173.5, 170.5, 169.4, 158.4, 142.6, 140.4, 140.3, 134.9, 131.4, 131.0, 127.0, 121.3, 119.4, 118.1, 109.4, 106.1, 82.6, 67.2, 55.1, 50.1, 48.8, 37.6, 35.1, 29.7, 28.4.

Step 1h. A solution of the compound from step 1g (215 mg, 0.40 mmol) in CH₂Cl₂ (5 mL) was treated with TFA (5 mL) at room temperature for 2.5 hours before being evaporated. The residue was chromatographed (silica, hexanes-EtOAc) to give the title compound (172 mg, 89%) as light yellow amorphous solid. ESIMS m/z=483.42 [M+H]⁺.

Step 1i. A solution of the compound from step 1g (10 mg) and CDI (20 mg) in DMF (1 mL) was stirred at 40° C. for 2.5 hours before charging methanesulfonamide (12 mg) and DBU (12.5 μL). The mixture was heated at 100° C. for another 10 hours before partition (EtOAc and water). The organics were washed (water, brine), dried (Na₂SO₄), and evaporated. The residue was chromatographed (silica, hexanes-EtOAc) to give the desired compound (7 mg). ESIMS m/z=560.36 [M+H]⁺.

### Example 2

### Compound of Formula (I), wherein M=Me, Q=4-tent-butyl-3-methoxyphenyl, Z=thiazol-2-ylmethyl, W═H, Y=—CH₂CH₂CH₂OMe, J=1H-pyrazol-1-ylmethyl

Step 2a. A solution of the compound from step 1g (50 mg, 0.093 mmol) in THF (1 mL) was treated with 9-BBN (0.5 M in THF, 0.3 mL) at room temperature for 12 hours before charging saturated NaHCO₃ (2 mL) and H₂O₂ (30% in water, 1 mL). The mixture was stirred at room temperature for another 3 hours before partition (water-EtOAc). The aqueous phase was extracted with EtOAc and the combined organics were washed (saturated NaHCO₃, water, brine), dried (Na₂SO₄), and evaporated. The residue was chromatographed (silica, hexanes-EtOAc) to give the desired compound containing some impurity. ESIMS m/z=557.24 [M+H]⁺.

Step 2b. A mixture of the compound from step 2a (10 mg, 18.0 mmol), tetrabutylammnium iodide (15 mg, 13.4 μmol) in MeI (0.2 mL) was treated with NaOH (50% in water, 1.0 mL) at room temperature for 2 hours before being partitioned (EtOAc-water). The organics were washed (water, brine), dried (Na₂SO₄), and evaporated. The residue was chromatographed (silica, hexanes-EtOAc) to give the desired compound (3 mg) as a colorless film. ESIMS m/z=571.22 [M+H]⁺.

Step 2c. A solution of the compound of step 2b (3 mg) in CH₂Cl₂ (0.5 mL) was treated with TFA (0.5 mL) at room temperature for 6 hours and the volatiles were removed by N₂ flow. The residue was chromatographed (silica, CH₂Cl₂-methanol) to give the desired compound (2 mg, 70%). ESIMS m/z=515.01 [M+H]⁺.

Step 2d. To a solution of the compound of step 2c (10.0 mg, 19.4 μmol) in CH₃CN (2 mL) was added CDI (31.5 mg, 0.194 mmol), the mixture was kept stirring until the disapperance of starting material. Methyl sulfonamide (22.1 mg, 0.233 mmol) and DBU (29.0 μL, 0.194 mmol) were added sequentially, and the reaction mixture was kept in a sealed tube and heated to 100° C. for 45 hours. The reaction was cooled down and the volatiles were evaporated. The residue was purified by flash column chromatography (silica, hexane-ethyl acetate) to give the title compound (2.9 mg, 25%) as a white solid. ESIMS m/z=591.96 [M+H]⁺.

### Example 3

### Compound of Formula (I), wherein M=Me, Q=4-tert-butyl-3-methoxyphenyl, Z=thiazol-2-ylmethyl, W═H, Y=—CH₂CH₂CH═NOMe, J=1H-pyrazol-1-ylmethyl

Step 3a. Into a solution of the compound from step 2a (95 mg, 0.17 mmol) in CH₂Cl₂ (5.0 mL) was added Dess-Martin periodinane (216 mg, 0.51 mmol), NaHCO₃ (250 mg) and t-BuOH (25 μmol). The resulted mixture was stirred at room temperature for 1.5 hours before dilution (EtOAc and saturated aqueous Na₂S₂O₃). The organics were washed (saturated NaHCO₃, water, brine), dried (Na₂SO₄), and evaporated. The residue was chromatographed (silica, hexanes-EtOAc) to give the desired compound (80 mg, 85%) as light yellow amorphous solid. ESIMS m/z=555.48 [M+H]⁺.

Step 3b. A solution of the compound from step 3a (6 mg) in CH₃CN (1 mL) was treated with O-methylhydroxylamine hydrochloride (10 mg) at room temperature for 12 hours before partition (EtOAc-saturated aqueous NaHCO₃). The organics were washed (saturated NaHCO₃, water, brine), dried (Na₂SO₄), and evaporated. The residue was chromatographed (silica, hexanes-EtOAc) to give the desired compound (3 mg). ESIMS m/z=584.12 [M+H]⁺.

Step 3c. The desired compound was prepared from the compound from step 3b following a similar procedure to that described in step 1h. ESIMS m/z=528.15 [M+H]⁺.

Step 3d. The title compound is prepared from the compound from step 3c following a similar procedure to that described in step 1i.

### Example 4

### Compound of Formula (I), wherein M=Me, Q=4-tert-butyl-3-methoxyphenyl, Z=thiazol-2-ylmethyl, W═H, Y=—CH₂CH₂CH₂NMe₂, J=1H-pyrazol-1-ylmethyl

Step 4a. Into a solution of the compound from step 3a (6 mg, 10 μmol) in CH₂Cl₂ (1 mL) was added dimethylamine (2 M in THF, 0.05 mL, 0.1 mmol), NaBH₃CN (8 mg, 0.1 mmol) and AcOH (1 drop). The resulted mixture was stirred at room temperature for 1 hour before being diluted with EtOAc and saturated aqueous NaHCO₃. The organics were washed (water, brine), dried (Na₂SO₄), and evaporated. The residue was chromatographed (silica, hexanes-EtOAc) to give the desired compound (4.4 mg). ESIMS m/z=584.25 [M+H]⁺.

Step 4b. The desired compound was prepared from the compound from step 4a following a similar procedure to that described in step 1h. ESIMS m/z=528.18 [M+H]⁺.

Step 4c. The title compound is prepared from the compound from step 4b following a similar procedure to that described in step 1i.

### Example 5

### Compound of Formula (I), wherein M=Me, Q=4-tert-butyl-3-methoxyphenyl, Z=thiazol-2-ylmethyl, W═H, Y=(E)-CH₂CH₇CH═CHCN, J=1H-pyrazol-1-ylmethyl

Step 5a. Into a solution of the compound from step 3a (15 mg) in toluene (2 mL) was treated with triphenylphosphoranylideneacetonitrile (25 mg) at room temperature for 12 hours before being diluted with EtOAc and saturated aqueous NaHCO₃. The organics were washed with water, brine, dried (Na₂SO₄), and evaporated. The residue was chromatographed (silica, hexanes-EtOAc) to give the desired compounds (14.5 mg, major isomer; and 5 mg, minor isomer). ESIMS m/z=578.19 [M+H]⁺, same for both isomers.

Step 5b. The desired compound was prepared from the compound from step 5a (major isomer) following a similar procedure to that described in step 1h. ESIMS m/z=522.12 [M+H]⁺.

Step 5c. The title compound is prepared from the compound from step 5b following a similar procedure to that described in step 1i.

### Example 6

### Compound of Formula (I), wherein M=Me, Q=4-tert-butyl-3-methoxyphenyl, Z=thiazol-2-ylmethyl, W═H, Y=(Z)-CH₂CH₂CH═CHCN, J=1H-pyrazol-1-ylmethyl

Step 6a. The desired compound was prepared from the compound from step 5a (minor isomer) following a similar procedure to that described in step 1h. ESIMS m/z=522.15 [M+H]⁺.

Step 6b. The title compound is prepared from the compound from step 6a following a similar procedure to that described in step 1i.

### Example 7

### Compound of Formula (I), wherein M=Me, Q=4-tert-butyl-3-methoxyphenyl, Z=thiazol-2-ylmethyl, W═H, Y=—CH₂CH₂CH₂CH₂CN, J=1H-pyrazol-1-ylmethyl

Step 7a. Into a solution of the compound from step 5a (major isomer, 10 mg) in i-PrOH (2 mL) and MeOH (0.1 mL) was treated with NaBH₄ (10 mg) at room temperature for 12 hours before being diluted with EtOAc and saturated aqueous NaHCO₃. The organics were washed with water, brine, dried (Na₂SO₄), and evaporated. The residue was chromatographed (silica, hexanes-EtOAc) to give the desired compound (9 mg). ESIMS m/z=580.12 [M+H]⁺.

Step 7b. The desired compound was prepared from the compound from step 7a following a similar procedure to that described in step 1h. ESIMS m/z=524.11 [M+H]⁺.

Step 7c. The title compound is prepared from the compound from step 7b following a similar procedure to that described in step 1i.

### Example 8

### Compound of Formula (I), wherein M=Me, Q=4-tert-butyl-3-methoxyphenyl, Z=thiazol-2-ylmethyl, W═H, Y=—CH₂CH₂CH═CF₂, J=1H-pyrazol-1-ylmethyl

Step 8a. Into a solution of the compound from step 3a (10 mg) in THF (1 mL) was added 12 drops of (Me₂N)₃P and 3 drops of CF₂Br₂ at −78° C. After being warmed to room temperature over 1 hour, the mixture was diluted with EtOAc and saturated aqueous NaHCO₃. The organics were washed with water, brine, dried (Na₂SO₄), and evaporated. The residue was chromatographed (silica, hexanes-EtOAc) to give the desired compound (10 mg). ESIMS m/z=589.14 [M+H]⁺.

Step 8b. The desired compound was prepared from the compound from step 8a following a similar procedure to that described in step 1h. ESIMS m/z=533.08 [M+H]⁺.

Step 8c. The title compound is prepared from the compound from step 8b following a similar procedure to that described in step 1i.

### Example 9

### Compound of Formula (I), wherein M=Me, Q=4-tert-butyl-3-methoxyphenyl, Z=thiazol-2-ylmethyl, W═H, Y=—CH₂CHO, J=1H-pyrazol-1-ylmethyl

Step 9a. A solution of the compound from step 1g (500 mg, 0.93 mmol) in THF (5 mL) and water (0.7 mL) was treated with NaIO₄ (397 mg, 1.86 mmol) in the presence of OsO₄ (4 wt %, 1.8 mL, 0.28 mmol) at room temperature for 5 hours before partition (EtOAc and water). The organics were washed with water and brine, dried (Na₂SO₄) and evaporated. The residue was chromatographed (silica, EtOAc-hexanes) to give the desired compound (347 mg, 60%). ESIMS m/z=541.16 [M+H]⁺.

Step 9b. A solution of the compound of step 9a (5 mg) in TFA (0.5 mL) was stirred at room temperature for 6 hours and the volatiles were removed by N₂ flow. The residue was chromatographed (silica, CH₂Cl₂-MeOH) to give the desired compound (4.5 mg). ESIMS m/z=485.09 [M+H]⁺.

Step 9c. The title compound is prepared from the compound from step 9b following a similar procedure to that described in step 1i.

### Example 10

### Compound of Formula (I), wherein M=Me, Q=4-tert-butyl 3-methoxyphenyl, Z=thiazol-2-ylmethyl, W═H, Y=—CH₂CH₂OMe, J=1H-pyrazol-1-ylmethyl

Step 10a. A solution of the compound from step 9a (100 mg, 0.18 mmol) in isopropanol (5 mL) and MeOH (0.25 mL) was treated with NaBH₄ (14 mg, 0.37 mmol) at room temperature for 2.5 hours before partition (EtOAc and water). The organics were washed with water and brine, dried (Na₂SO₄) and evaporated. The residue was chromatographed (silica, EtOAc-hexanes) to give the desired compound mixed with some impurity (84 mg). ESIMS m/z=543.20 [M+H]⁺.

Step 10b. A solution of the compound from step 10a (10 mg) in MeI (1 mL) was treated with aqueous NaOH (50%, 1 mL) in the presence of n-Bu₄NI (2 mg) at room temperature for 20 hours before partition (EtOAc and water). The organics were washed (water and brine), dried (Na₂SO₄) and evaporated. The residue was chromatographed (silica, EtOAc-hexanes) to give the desired compound. ESIMS m/z=557.14 [M+H]⁺.

Step 10c. The desired compound (3.1 mg) was obtained from the compound of step 10b using similar procedures to that described in step 9b. ESIMS m/z=501.16 [M+H]⁺.

Step 10d. The title compound is prepared from the compound from step 10c following a similar procedure to that described in step 1i.

### Example 11

### Compound of Formula (I), wherein M=Me, Q=4-tent-butyl-3-methoxyphenyl, Z=thiazol-2-ylmethyl, W═H, Y=(E)-CH₂CH═CHCN, J=1H-pyrazol-1-ylmethyl

Step 11a. A solution of the compound from step 9a (20 mg, 0.04 mmol) in toluene (1 mL) was treated with triphenylphosphoranylideneacetonitrile (22 mg, 0.07 mmol) at room temperature for 12 hours before being chromatographed (silica, EtOAc-hexanes) to give the desired compounds (13.4 mg, major isomer; and 3.3 mg, minor isomer). ESIMS m/z=564.04 [M+H]⁺ for both isomers.

Step 11b. A solution of the compound of step 11a (major isomer, 5 mg) in CH₂Cl₂ (0.5 mL) was treated with TFA (0.5 mL) at room temperature for 3 hours and the volatiles were removed by N₂ flow. The residue was chromatographed (silica, CH₂Cl₂-MeOH) to give the desired compound (4 mg). ESIMS m/z=508.14 [M+H]⁺.

Step 11c. The title compound is prepared from the compound from step 11b following a similar procedure to that described in step 1i.

### Example 12

### Compound of Formula (I), wherein M=Me, Q=4-tent-butyl-3-methoxyphenyl, Z=thiazol-2-ylmethyl, W═H, Y=(Z)-CH₂CH═CHCN, J=1H-pyrazol-1-ylmethyl

Step 12a. The desired compound (1.1 mg) was obtained from the compound of step 11a (minor isomer, 3.3 mg) using similar procedures to that described in step 11b. ESIMS m/z=508.10 [M+H]⁺.

Step 12b. The title compound is prepared from the compound from step 12a following a similar procedure to that described in step 1i.

### Example 13

### Compound of Formula (I), wherein M=Me, Q=4-tent-butyl-3-methoxyphenyl, Z=thiazol-2-ylmethyl, W═H, Y=(E)-CH₂CH₇CH₇CN, J=1H-pyrazol-1-ylmethyl

Step 13a. A solution of the compound from step 11a (major isomer, 8 mg) in MeOH (1 mL) was treated with excess NaBH₄ at room temperature for 2 hours before charging tris(hydroxymethyl)aminomethane, EtOAc and water. The organics were washed with water and brine, dried (Na₂SO₄) and evaporated. The residue was chromatographed (silica, EtOAc-hexanes) to give the desired compound mixed with some impurity. ESIMS m/z=566.23 [M+H]⁺.

Step 13b. The desired compound (2.2 mg) was obtained from the compound of step 13a using similar procedures to that described in step 11b. ESIMS m/z=510.17 [M+H]⁺.

Step 13c. The title compound is prepared from the compound from step 13b following a similar procedure to that described in step 1i.

### Example 14

### Compound of Formula (I), wherein M=Me, Q=4-tent-butyl-3-methoxyphenyl, Z=thiazol-2-ylmethyl, W═H, Y=—CH₂CH═NOMe, J=1H-pyrazol-1-ylmethyl

Step 14a. A solution of the compound from step 9a (10 mg) in MeCN (1 mL) was treated with excess O-methylhydroxylamine hydrochloride at room temperature for 20 hours before partition (EtOAc-water). The organics were washed with water and brine, dried (Na₂SO₄) and evaporated. The residue was chromatographed (silica, EtOAc-hexanes) to give the desired compound (6.0 mg) as a 1.5:1 isomeric mixture. ESIMS m/z=570.19 [M+H]⁺.

Step 14b. The desired compound (3.1 mg) was obtained from the compound of step 14a (6.0 mg) using similar procedures to that described in step 9b. ESIMS m/z=514.15 [M+H]⁺.

Step 14c. The title compound is prepared from the compound from step 14b following a similar procedure to that described in step 1i.

### Example 15

### Compound of Formula (I), wherein M=Me, Q=4-tent-butyl-3-methoxyphenyl, Z=thiazol-2-ylmethyl, W═H, Y=—CH₂CH₂NMe₂, J=1H-pyrazol-1-ylmethyl

Step 15a. A solution of the compound from step 9a (5 mg) in MeCN (1 mL) was treated with dimethylamine (2 M in THF, 0.5 mL) and excess NaBH₃CN in the presence of HOAc (0.7 mL) at room temperature for 20 hours before partition (EtOAc-water). The organics were washed with water and brine, dried (Na₂SO₄) and evaporated. The residue was chromatographed (silica, EtOAc-hexanes) to give the desired compound (4.3 mg). ESIMS m/z=570.23 [M+H]⁺.

Step 15b. The desired compound (4.2 mg) was obtained from the compound of step 15a (4.3 mg) using similar procedures to that described in step 9b. ESIMS m/z=514.18 [M+H]⁺.

Step 15c. The title compound is prepared from the compound from step 15b following a similar procedure to that described in step 1i.

### Example 16

### Compound of Formula (I), wherein M=Me, Q=4-tent-butyl-3-methoxyphenyl, Z=thiazol-2-ylmethyl, W═H, Y=—CH₂CH═CH₂, J=1H-pyrazol-1-ylmethyl

The title compound was prepared from the compound from step 1h and cyclopropylsulfonamide following a similar procedure to that described in step 1i. ESIMS m/z=586.61 [M+H]⁺.

### Example 17

### Compound of Formula (I), wherein M=Me, Q=4-tent-butyl-3-methoxyphenyl, Z=thiazol-2-ylmethyl, W═H, Y=—CH₂CH═CH₂, J=1H-pyrazol-1-ylmethyl

The title compound was prepared from the compound from step 1h and phenylsulfonamide following a similar procedure to that described in step 1i. ESIMS m/z=622.53 [M+H]⁺.

### Example 18

### Compound of Formula (I), wherein M=-Ph, Q=4-tent-butyl-3-methoxyphenyl, Z=thiazol-2-yl, W═H, Y=—CH₂CH₂CN, J=1H-pyrazol-1-ylmethyl

Step 18a. A solution of the compound from step 3a (0.19 mmol) in dioxane (5 mL) was treated with hydroxylamine hydrochloride (50 mg) at room temperature for 26 hours before being diluted with EtOAc and saturated aqueous NaHCO₃. The organics were washed (saturated NaHCO₃, water, brine), dried (Na₂SO₄), and evaporated. The residue was chromatographed (silica, CH₂Cl₂-MeOH) to give the desired compound (113 mg). ESIMS m/z=570.47 [M+H]⁺.

Step 18b. A solution of the compound from step 18a (110 mg) in pyridine (3.8 mL) was treated with acetic anhydride (0.054 mL) under reflux for 24 hours before being concentrated. The residue was chromatographed (silica, hexane-EtOAc) to give the desired compound (50 mg). ESIMS m/z=552.44 [M+H]⁺.

Step 18c. The desired compound was prepared from the compound from step 18b following a similar procedure to that described in step 9b. ESIMS m/z=496.39 [M+H]⁺.

Step 18d. The title compound is prepared from the compound from step 18c and cyclopropylsulfonamide following a similar procedure to that described in step 1i.

### Example 19

### Compound of Formula (I), wherein M=-Ph, Q=4-tert-butyl-3-methoxyphenyl, Z=thiazol-2-yl, W═H, Y=—CH₂CN, J=1H-pyrazol-1-ylmethyl

Step 19a. The desired compound was prepared from the compound from step 9a following a similar procedure to that described in step 18a. ESIMS m/z=556.44 [M+H]⁺.

Step 19b. The desired compound was prepared from the compound from step 19a following a similar procedure to that described in step 18b. ESIMS m/z=538.44 [M+H]⁺.

Step 19c. The desired compound was prepared from the compound from step 19b following a similar procedure to that described in step 9b. ESIMS m/z=482.37 [M+H]⁺.

Step 19d. The title compound is prepared from the compound from step 19c following a similar procedure to that described in step 1i.

### Example 20

### Compound of Formula (I), wherein M=-Ph, Q=4-tert-butyl-3-methoxyphenyl, Z=thiazol-2-yl, W═H, Y=—CH₂CH₂-(tetrazol-5-yl), J=1H-pyrazol-1-ylmethyl

Step 20a. Into a solution of the compound from step 18b (17 mg) in i-PrOH (1.5 mL) was added water (1.5 mL), ZnBr₂ (21 mg) and NaN₃ (24 mg). The mixture was refluxed for 24 hours before being partitioned (water and EtOAc). The organics were washed (water, brine), dried (Na₂SO₄), and evaporated. The residue was chromatographed (silica, CH₂Cl₂-MeOH) to give the desired compound (2.2 mg). ESIMS m/z=595.53 [M+H]⁺. ¹HNMR (CD₃OD, 500 MHz) 7.82 (s, 1H), 7.64 (s, 1H), 7.53 (d, 1H), 7.46 (d, 1H), 7.25 (d, 1H), 7.02 (s, 1H), 6.98 (m, 1H), 6.40 (s, 1H), 5.22 (d, 1H), 4.46 (d, 1H), 3.72 (s, 3H), 3.68 (m, 1H), 3.38 (m, 1H), 2.96 (br, 1H), 2.45 (m, 3H), 1.60 (s, 9H), 1.38 (s, 9H).

Step 20b. The desired compound was prepared from the compound from step 20a following a similar procedure to that described in step 12b. ESIMS m/z=539.46 [M+H]⁺. ¹HNMR (CD₃OD, 500 MHz) 7.68 (s, 1H), 7.64 (s, 1H), 7.56 (d, 1H), 7.48 (d, 1H), 7.16 (d, 1H), 6.75 (s, 1H), 6.73 (m, 1H), 6.34 (s, 1H), 5.38 (d, 1H), 4.75 (m, 1 h, 4.60 (d, 1H), 4.14 (d, 1H), 3.57 (s, 3H), 3.65 (m, 1H), 2.80 (m, 1H), 2.55 (m, 1H), 2.20 (m, 1H), 1.25 (s, 9H).

Step 20c. The title compound is prepared from the compound from step 20b following a similar procedure to that described in step 1i.

### Example 21

### Compound of Formula (I), wherein M=-Ph, Q=4-tent-butyl-3-methoxyphenyl, Z=thiazol-2-yl, W═H, Y=—CH₂-(tetrazol-5-yl), J=1H-pyrazol-1-ylmethyl

Step 21a. The desired compound was prepared from the compound from step 19b following a similar procedure to that described in step 20a. ESIMS m/z=581.51 [M+H]⁺.

Steps 21b and 21c. The title compound is prepared from the compound from step 21a following similar procedures to that described in steps 20b and 20c.

The title compounds of examples 22-57 were/may be prepared using similar procedures described in examples 1-21 and 58-81.

### Example 22

### Compound of Formula (I), wherein M=—NH₂, Q=4-tent-butyl-3-methoxyphenyl, Z=thiazol-2-ylmethyl, W═H, Y=—CH₂CH₂CH₂OMe, J=1H-pyrazol-1-ylmethyl

### Example 23

### Compound of Formula (I), wherein M=—NHPh, Q=4-tent-butyl-3-methoxyphenyl, Z=thiazol-2-ylmethyl, W═H, Y=—CH₂CH₂CH₂OMe, J=1H-pyrazol-1-ylmethyl

### Example 24

### Compound of Formula (I), wherein M=—NMe₂, Q=4-tert-butyl-3-methoxyphenyl, Z=thiazol-2-ylmethyl, W═H, Y=—CH₂CH₂CH₂OMe, J=1H-pyrazol-1-ylmethyl

ESIMS m/z=620.93 [M+H]⁺.

### Example 25

### Compound of Formula (I), wherein M=-Ph, Q=4-tert-butyl-3-methoxyphenyl, Z=thiazol-2-ylmethyl, W═OH, Y=—CH₂CH₂CH₂OMe, J=1H-pyrazol-1-ylmethyl

### Example 26

### Compound of Formula (I), wherein M=-Phenyl-fluoro-(p), Q=4-tert-butyl-3-methoxyphenyl, Z=thiazol-2-ylmethyl, W═H, Y=—CH₂CH₇CH₂OMe, J=1H-pyrazol-1-ylmethyl

ESIMS m/z=672.23 [M+H]⁺.

### Example 27

### Compound of Formula (I), wherein M=-Phenyl-fluoro-(m), Q=4-tert-butyl-3-methoxyphenyl, Z=thiazol-2-ylmethyl, W═H, Y=—CH₂CH₇CH₂OMe, J=1H-pyrazol-1-ylmethyl

ESIMS m/z=672.22 [M+H]⁺.

### Example 28

### Compound of Formula (I), wherein M=-Phenyl-fluoro-(O), Q=4-tert-butyl-3-methoxyphenyl, Z=thiazol-2-ylmethyl, W═H, Y=—CH₂CH₇CH₂OMe, J=1H-pyrazol-1-ylmethyl

ESIMS m/z=672.22 [M+H]⁺.

### Example 29

### Compound of Formula (I), wherein M=-2-pyridyl, Q=4-tert-butyl-3-methoxyphenyl, Z=thiazol-2-ylmethyl, W═H, Y=—CH₂CH₂CH₂OMe, J=1H-pyrazol-1-ylmethyl

### Example 30

### Compound of Formula (I), wherein M=-3-pyridyl, Q=4-tert-butyl-3-methoxyphenyl, Z=thiazol-2-ylmethyl, W═H, Y=—CH₂CH₂CH₂OMe, J=1H-pyrazol-1-ylmethyl

### Example 31

### Compound of Formula (I), wherein M=-4-pyridiyl, Q=4-tert-butyl-3-methoxyphenyl, Z=thiazol-2-ylmethyl, W═H, Y=—CH₂CH₂CH₂OMe, J=1H-pyrazol-1-ylmethyl

### Example 32

### Compound of Formula (I), wherein M=-Ph, Q=4-tent-butyl-3-trifluoromethoxyphenyl, Z=thiazol-2-ylmethyl, W═H, Y=—CH₂CH₂CH₂OMe, J=1H-pyrazol-1-ylmethyl

ESIMS m/z=653.97 [M+H]⁺.

### Example 33

### Compound of Formula (I), wherein M=-Ph, Q=5-tert-butyl-4-methoxypyridin-2-yl, Z=thiazol-2-ylmethyl, W═H, Y=—CH₂CH₂CH₂OMe, J=1H-pyrazol-1-ylmethyl

### Example 34

### Compound of Formula (I), wherein M=-Ph, Q=4-tert-butyl-3-vinylphenyl, Z=thiazol-2-ylmethyl, W═H, Y=—CH₂CH₂CH₇OMe, J=1H-pyrazol-1-ylmethyl

### Example 35

### Compound of Formula (I), wherein M=-Ph, Q=4-tert-butyl-3-bromophenyl, Z=thiazol-2-ylmethyl, W═H, Y=—CH₂CH₂CH₂OMe, J=1H-pyrazol-1-ylmethyl

### Example 36

### Compound of Formula (I), wherein M=-Ph, Q=4-tert-butyl-3-chlorophenyl, Z=thiazol-2-ylmethyl, W═H, Y=—CH₂CH₂CH₇OMe, J=1H-pyrazol-1-ylmethyl

### Example 37

### Compound of Formula (I), wherein M=-Ph, Q=4-tent-butyl-3-fluorophenyl, Z=thiazol-2-ylmethyl, W═H, Y=—CH₂CH₂CH₂OMe, J=1H-pyrazol-1-ylmethyl

### Example 38

### Compound of Formula (I), wherein M=-Ph, Q=4-tert-butyl-5-bromo-2-fluorophenyl, Z=thiazol-2-ylmethyl, W═H, Y=—CH₂CH₂CH₂OMe, J=1H-pyrazol-1-ylmethyl

### Example 39

### Compound of Formula (I), wherein M=-Ph, Q=4-tert-butyl-5-bromo-2-fluorophenyl, Z=(5-methyl-isoxazol-3-yl)methyl, W═H, Y=—CH₂CH₂CH₂OMe, J=1H-pyrazol-1-ylmethyl

### Example 40

### Compound of Formula (I), wherein M=-Ph, Q=4-tert-butyl-5-bromo-2-fluorophenyl, Z=thiazol-2-ylmethyl, W═H, Y=—CH₂CH₂CH₂OMe, J=1,3-thiazol-4-ylmethyl

### Example 41

### Compound of Formula (I), wherein M=-Ph, Q=4-tert-butyl-5-bromo-2-fluorophenyl, Z=thiazol-2-ylmethyl, W═H, Y=—CH₂CH₂CH₂OMe, J=1,3-thiazol-2-ylmethyl

### Example 42

### Compound of Formula (I), wherein M=-Ph, Q=4-tert-butyl-5-bromo-2-fluorophenyl, Z=thiazol-2-ylmethyl, W═H, Y=—CH₂CH₂CH₂OMe, J=isothiazol-3-ylmethyl

### Example 43

### Compound of Formula (I), wherein M=-Ph, Q=4-tert-butyl-5-bromo-2-fluorophenyl, Z=Bn, W═H, Y=—CH₂CH₂CH₇OMe, J=isothiazol-3-ylmethyl

### Example 44

### Compound of Formula (I), wherein M=-Ph, Q=4-tert-butyl-5-bromo-2-fluorophenyl, Z=thiazol-2-ylmethyl, W═H, Y=—CH₂CH₂CH₂OMe, J=Bn

### Example 45

### Compound of Formula (I), wherein M=-Ph, Q=4-tent-butyl-3-methoxyphenyl, Z=thiazol-2-ylmethyl, W═H, Y=propargyl, J=1H-pyrazol-1-ylmethyl

### Example 46

### Compound of Formula (I), wherein M=-Ph, Q=4-tent-butyl-3-methoxyphenyl, Z=thiazol-2-ylmethyl, W═H, Y=—CH₂CH₂CH₂OMe, J=—CH₂CH₂OMe

### Example 47

### Compound of Formula (I), wherein M=-Ph, Q=4-tent-butyl-3-methoxyphenyl, Z=thiazol-2-ylmethyl, W═H, Y=—CH₂CH₂CH₂OMe, J=—CH₂CH₂NMe₂

### Example 48

### Compound of Formula (I), wherein M=-Ph, Q=4-tent-butyl-3-methoxyphenyl, Z=2-(thiazol-2-yl)ethyl, W═H, Y=—CH₂CH₇CH₇OMe, J=—CH₂CH₂NMe₂

### Example 49

### Compound of Formula (I), wherein M=2,4-difluorophenyl, Q=4-tert-butyl-3-methoxyphenyl, Z=thiazol-2-ylmethyl, W═H, Y=—CH₂CH₇CH₂OMe, J=1H-pyrazol-1-ylmethyl

ESIMS m/z=690.22 [M+H]⁺.

### Example 50

### Compound of Formula (I), wherein M=tert-butyl, Q=4-tent-butyl-3-methoxyphenyl, Z=thiazol-2-ylmethyl, W═H, Y=—CH₂CH₂CH₂OMe, J=1H-pyrazol-1-ylmethyl

ESIMS m/z=633.94 [M+H]⁺.

### Example 51

### Compound of Formula (I), wherein M=cyclopropyl, Q=4-tent-butyl-3-methoxyphenyl, Z=thiazol-2-ylmethyl, W═H, Y=—CH₂CH₂CH₂OMe, J=1H-pyrazol-1-ylmethyl

ESIMS m/z=617.91 [M+H]⁺.

### Example 52

### Compound of Formula (I), wherein M=trifluoromethyl, Q=4-tent-butyl-3-methoxyphenyl, Z=thiazol-2-ylmethyl, W═H, Y=—CH₂CH₂CH₂OMe, J=1H-pyrazol-1-ylmethyl

ESIMS m/z=645.86 [M+H]⁺.

### Example 53

### Compound of Formula (I), wherein M=2,6-difluorophenyl, Q=4-tert-butyl-3-methoxyphenyl, Z=thiazol-2-ylmethyl, W═H, Y=—CH₂CH₇CH₂OMe, J=1H-pyrazol-1-ylmethyl

ESIMS m/z=689.86 [M+H]⁺.

### Example 54

### Compound of Formula (I), wherein M=2-chlorophenol, Q=4-tert-butyl-3-methoxyphenyl, Z=thiazol-2-ylmethyl, W═H, Y=—CH₂CH₂CH₂OMe, J=1H-pyrazol-1-ylmethyl

ESIMS m/z=687.85 [M+H]⁺.

### Example 55

### Compound of Formula (I), wherein M=2-cyanophenyl, Q=4-tert-butyl-3-methoxyphenyl, Z=thiazol-2-ylmethyl, W═H, Y=—CH₂CH₂CH₂OMe, J=1H-pyrazol-1-ylmethyl

ESIMS m/z=678.88 [M+H]⁺.

### Example 56

### Compound of Formula (I), wherein M=2-trifluoromethylphenyl, Q=4-tent-butyl-3-methoxyphenyl, Z=thiazol-2-ylmethyl, W═H, Y=—CH₂CH₂CH₂OMe, J=1H-pyrazol-1-ylmethyl

ESIMS m/z=721.86 [M+H]⁺.

### Example 57

### Compound of Formula (I), wherein M=2-trifluoromethoxyphenyl, Q=4-tent-butyl-3-methoxyphenyl, Z=thiazol-2-ylmethyl, W═H, Y=—CH₂CH₂CH₂OMe J=1H-pyrazol-1-ylmethyl

ESIMS m/z=737.84 [M+H]⁺.

### Example 58

### Compound of Formula (I), wherein M=2-fluorophenyl, Q=4-tert-butyl-2-fluoro-3-methoxyphenyl, Z=thiazol-2-ylmethyl, W═H, Y=—CH₂CH₂CH₂OMe, J=1H-pyrazol-1-ylmethyl

Step 58a. To a stirred solution of compound from step 1f (2 g, 8.85 mmol) in dichloromethane (10 mL) was added tert-butylamine (1.87 mL, 17.7 mmol). The resulting mixture was stirred at room temperature for 1 hour before being partitioned between water and EtOAc. The organic phase was dried (sodium sulfate), evaporated and the residue was chromatographed (silica, hexanes-EtOAc) to give the desired compound (2.27 g, 98%). ESIMS m/z=264.16 [M+H]⁺.

Step 58b. To a stirred solution of compound from step 58a (1.7 g, 6.5 mmol) in THF (60 mL) was added TMEDA (2.44 mL, 16.7 mmol) and sec-butyl lithium (11.6 mL, 1.4 M in cyclohexane, 16.7 mmol) at −78° C. The resulting mixture was stirred at −78° C. for 1 hour before being added N-fluorobenzenesulfonimide (5.12 g, 16.7 mmol) in THF (10 mL). The mixture was stirred at −78° C. for another 4 hours before being quenched with aqueous NH₄Cl. The resulting slurry was partition between water and Et₂O. The organic phase was dried (sodium sulfate), evaporated and the residue was chromatographed (silica, hexanes-EtOAc) to give the desired compounds as a monofluorinated regio isomeric mixture (1.4 g, 78%). ESIMS m/z=282.19 [M+H]⁺.

Step 58c. To a stirred solution of compound from step 58b (1.4 g, 5 mmol) in acetonitrile (25 mL) was added sodium phosphate dibasic (1.1 g, 7.5 mmol) and trimethyloxonium tetrafluoroborate (2.3 g, 15 mmol). The resulting mixture was stirred at room temperature for 3.5 hours before being added aqueous NaHCO₃. The mixture was stirred at room temperature for another 12 hours before being partitioned between water and EtOAc. The organic phase was dried (sodium sulfate), evaporated and the residue was chromatographed (silica, hexanes-EtOAc) to give the desired compounds as a mixture (500 mg, 31%).

Step 58d. To a stirred solution of compound from step 58c (500 mg, 2.21 mmol) in methanol (25 mL) was added sodium hydroxide (50% in water, 1.77 g, 22.1 mmol). The resulting mixture was refluxed at 85° C. for 3 hour before being partitioned between aqueous hydrochloric acid (1M) and EtOAc. The organic phase was dried (sodium sulfate), evaporated and the residue was chromatographed (silica, hexanes-EtOAc) to give the desired compounds as a mixture (220 mg, 47%).

Step 58e. The stirred solution of compound from step 58d (220 mg, 1 mmol) in thionyl chloride (4 mL) was refluxed at 85° C. for 2 hours before all volatiles were removed by rotavap. The resulting slurry was charge with dichloromethane (1.5 mL). The mixture was added compound from step 62b (315 mg, 0.83 mmol) and triethylamine (0.8 mL). The resulting mixture was stirred at room temperature for 17 hours before being partitioned between water and EtOAc. The organic phase was dried (sodium sulfate), evaporated and the residue was purified by HPLC (C-18, acetonitrile-20 mM NH₄HCO₃ in water) to provide the desired compound (62 mg, 12%). ESIMS m/z=589.15 [M+H]⁺.

Step 58f. The desired compound was prepared from the compound from step 58e following a similar procedure to that described in step 1h. ESIMS m/z=533.04 [M+H]⁺.

Step 58g. The title compound was prepared from the compound from step 58f following a similar procedure to that described in step 1i. ESIMS m/z=689.87 [M+H]⁺.

### Example 59

### Compound of Formula (I), wherein M=2-fluorophenyl, Q=4-tent-butyl-2,6-difluoro-3-methoxyphenyl, Z=thiazol-2-ylmethyl, W═H, Y=—CH₂CH₂CH₂OMe, J=1H-pyrazol-1-ylmethyl

Step 59a. To a stirred solution of compound from step 58b (1.02 g, 3.63 mmol) in THF (50 mL) was added TMEDA (2.82 mL, 18 mmol) and sec-butyl lithium (13.1 mL, 1.4 M in cyclohexane, 18 mmol). at −78° C. The resulting mixture was stirred at −78° C. for 1 hour before being added N-fluorobenzenesulfonimide (5.72 g, 18 mmol) in THF (10 mL). The mixture was stirred at −78° C. for another 2 hours before being quenched with aqueous NH₄Cl. The resulting slurry was partition between water and Et₂O. The organic phase was dried (sodium sulfate), evaporated and the residue was chromatographed (silica, hexanes-EtOAc) to give the desired compound (650 mg, 60%). ESIMS m/z=300.04 [M+H]⁺.

Step 59b. To a stirred solution of compound from step 59a (350 mg, 2.32 mmol) in dioxane (10 mL) was added perchloric acid (70% in water, 4.3 mL) and water (5.7 mL). The resulting mixture was refluxed at 120° C. for 12 hours before being partitioned between aqueous NaOH (2M) and EtOAc. The aqueous phase was separated, acidified with hydrochloric acid (1N) and extracted with EtOAc. The extract was dried (sodium sulfate), evaporated and the residue was chromatographed (silica, hexanes-EtOAc) to give the desired compound (180 mg, 63%).

Step 59c. The desired compound was prepared from the compound from step 59b following a similar procedure to that described in step 58e. ESIMS m/z=606.94 [M+H]⁺.

Step 59d. The desired compound was prepared from the compound from step 59c following a similar procedure to that described in step 1h. ESIMS m/z=551.02 [M+H]⁺.

Step 59e. The title compound was prepared from the compound from step 59d following a similar procedure to that described in step 1i. ESIMS m/z=708.00 [M+H]⁺

### Example 60

### Compound of Formula (I), wherein M=methyl, Q=4-tert-butyl-3-methoxyphenyl, Z=pyridin-2-ylmethyl, W═H, Y=—CH₂CH₂CN, J=1H-pyrazol-1-ylmethyl

Step 60a. Into a solution of the compound from step 1d (14.50 g, 47.34 mmol) in acetonitrile (200 mL) and H₂O (15 mL) cooled with a water bath was added benzyltriethylammonium chloride (0.323 g, 1.42 mmol) and potassium carbonate (13.09 g, 94.68 mmol). Acrylonitrile (3.43 mL, 52.07 mmol) was added dropwisely. The mixture was vigorously stirred at room temperature for 30 minutes before being concentrated. The residue was taken up in ethyl acetate and water. The organic layer was washed with brine, dried (Na₂SO₄) and evaporated to afford a light brwon oil (17.53 g), which was used directly at the next step. ESIMS m/z=360.13 [M+H]⁺.

Step 60b. To a solution of the crude product from step 60a (47.34 mmol at most) in EtOH (200 mL) containing bromocresol green (2 mg) at 0° C. was added NaBH₄ (2.686 g, 71.01 mmol). After 20 minutes at 0° C., MeOH (150 mL) was added, followed by more NaBH₄ (2.686 g, 71.01 mmol). The reaction mixture was stirred at room temperature while more NaBH₄ and glacial acetic acid were added portionwisely to maintain the pH ˜5 until the reaction was complete. More acetic acid was added to quench the reaction. It was basicified with saturated NaHCO₃ solution before being concentrated. The residue was taken up in ethyl acetate and water. The organic layer was washed with brine, dried (Na₂SO₄) and evaporated. The residue was chromatographed (silica, hexanes-EtOAc with 1% Et₃N) to give the desired compound (8.70 g, 51% over 2 steps) as a light yellow oil. ESIMS m/z=362.15 [M+H]⁺.

Step 60c. To a solution of the crude product from step 60a (10.35 mmol at most) in THF (60 mL) was added 1N HCl (21 mL) at room temperature. It was stirred at room temperature for 2.5 hours before THF was evaporated off. The remaining aqueous solution was extracted with ether. The aqueous layer was basicified with K₂CO₃ before being extracted with CH₂Cl₂ and EtOAc. The combined organics were washed (brine), dried (Na₂SO₄), and evaporated to affored the desired compound (2.636 g, 96% over 2 steps) as an orange oil. ESIMS m/z=265.20 [M+H]⁺.

Step 60d. To a solution of the compound from step 60c (0.200 g, 0.757 mmol) and pyridine-2-carboxaldehyde (81.8 mg, 0.764 mmol) in CH₂Cl₂ (1.5 mL) was added MgSO₄ (0.455 g, 3.78 mmol) at rt. The suspension was stirred at room temperature for 2 hours. More pyridine-2-carboxaldehyde (41.0 mg, 0.382 mmol) in CH₂Cl₂ (0.5 mL) was added. After 1 hour, the suspension was filtered. The filtrate was concentrated to afford the desired compound as a yellow oil, which was used directly for next step. ESIMS m/z=354.27 [M+H]⁺.

Step 60e. To a solution of the compound from step 60d in MeOH (4 mL) containing bromocresol green (2 mg) at 0° C. was added glacial acetic acid until the solution turned bright yellow. NaBH₄ (57.2 mg, 1.51 mmol) was added at 0° C. The mixture was stirred at room temperature while more NaBH₄ and then glacial acetic acid were added portionwise to maintain the pH ˜5 until the reaction was complete. More acetic acid was added to quench the reaction. The reaction mixture was basicified with K₂CO₃ before being concentrated. The residue was taken up in ethyl acetate and water. The organic layer was washed with brine, dried (Na₂SO₄) and evaporated. The residue was chromatographed (silica, hexanes-EtOAc with 1% Et₃N) to give the desired compound (0.252 g, 93% over 2 steps) as a yellow oil. ESIMS m/z=356.29 [M+H]⁺.

Step 60f. A solution of the compound from step 60e (65.0 mg, 0.183 mmol) in CH₂Cl₂ (0.18 mL) was treated with Et₃N (0.15 mL) and the compound of step 1f (124.5 mg, 0.549 mmol) at room temperature overnight. MeOH (0.1 mL) was added to quench the reaction. After 10 minutes at room temperature, the mixture was concentrated. The residue was chromatographed (silica, hexanes-EtOAc) to give the desired compound (78.2 mg, 78%) as an off-white solid. ESIMS m/z=546.39 [M+H]⁺.

Step 60g. A solution of the compound from step 60f (76.0 mg, 0.139 mmol) in CH₂Cl₂ (0.5 mL) was treated with TFA (2.5 mL) at room temperature for 7 hours before being evaporated. The residue was evaporated with CH₂Cl₂ before being purified by HPLC (C-18, acetonitrile-20 mM NH₄HCO₃ in water) to give the desired compound (16.0 mg, 23%) as a white powder. ESIMS m/z=490.33[M+H]⁺.

Step 60h. The title compound is prepared from the compound from step 60g following a similar procedure to that described in step 1i.

### Example 61

### Compound of Formula (I), wherein M=methyl, Q=4-tert-butyl-3-methoxyphenyl, Z=(5-methylisoxazol-3-yl)methyl, W═H, Y=—CH₂CH₇CN, J=1H-pyrazol-1-ylmethyl

Step 61a. To a solution of the compound from step 60c (0.227 g, 0.859 mmol) and 5-methylisoxazole-3-carboxaldehyde (0.143 g, 1.29 mmol) in CH₂Cl₂ (2 mL) was added MgSO₄ (0.516 g, 4.29 mmol). The suspension was stirred at room temperature for 2 hours before being filtered. The filtrate was concentrated to affore the desired compound as a yellow oil, which was used directly for next step. ESIMS m/z=358.24 [M+H]⁺.

Step 61b. The desired compound (0.227 g, 74% over 2 steps) as a yellow oil was prepared from the compound from step 61a (0.859 mmol at most) following a similar procedure to that described in step 60e. ESIMS m/z=360.19 [M+H]⁺.

Step 61c. The desired compound (73.5 mg, 58%) as a yellow sticky oil was prepared from the compound from step 61b (83.0 mg, 0.231 mmol) following a similar procedure to that described in step 60f. ESIMS m/z=550.41 [M+H]⁺.

Step 61d. The desired compound (20.4 mg, 32%) as a white powder was prepared from the compound from step 61c (76.0 mg, 0.139 mmol) following a similar procedure to that described in step 60g. ESIMS m/z=494.19 [M+H]⁺.

Step 61e. The title compound is prepared from the compound from step 61d following a similar procedure to that described in step 1i.

### Example 62

### Compound of Formula (Ia), wherein M=2-fluorophenyl, Q=5-bromo-4-tert-butyl-2-fluorophenyl, Z=pyridin-2-ylmethyl, W═H, Y=—CH₂CH₇CH₂OMe, J=1H-pyrazol-1-ylmethyl

Step 62a. To a solution of the compound from step 1d (0.124 g, 0.402 mmol) in DMSO (4 mL) was added NaH (95%, 10.7 mg, 0.445 mmol) at room temperature. After 10 minutes, a solution of 1-iodo-3-methoxypropane (88.5 mg, 0.442 mmol) in DMSO (0.5 mL) was added dropwisely. The mixture was stirred at room temperature for 30 minutes before being quenched with saturated NaHCO₃ solution. The residue was partitioned (EtOAc and water). The organic layer was washed with brine, dried (Na₂SO₄), and evaporated to afford a yellow sticky oil (0.146 g), which was used directly for next step. ESIMS m/z=379.13 [M+H]⁺.

Step 62b. To a solution of the compound from step 62a in MeOH (4 mL) containing bromocresol green (2 mg) at 0° C. was added glacial acetic acid until the solution turned bright yellow. NaBH₄ (29.2 mg, 0.771 mmol) was added at 0° C. The reaction mixture was stirred at room temperature while more NaBH₄ and then glacial acetic acid added portionwisely to maintain the pH ˜5 until the reaction was complete. More acetic acid was added to quench the reaction. The reaction mixture was basicified with K₂CO₃ before being concentrated. The residue was taken up in ethyl acetate and water. The organic layer was washed with brine, dried (Na₂SO₄) and evaporated. The residue was chromatographed (silica, CH₂Cl₂-EtOAc with 1% Et₃N) to give the desired compound (0.113 g, 74% over 2 steps) as a yellow oil. ESIMS m/z=381.14 [M+H]⁺.

Step 62c. The desired product was prepared from the compound of step 62b following a procedure similar to that described in step 60c. ESI MS m/z=284.16 [M+H]⁺.

Step 62d. To a solution of the compound from step 62c (2.000 g, 7.058 mmol) and (1S, 2S,5S)-(+2-hydroxy-3-pinone (1.425 g, 8.469 mmol) in toluene (18 mL) was added BF₃.Et₂O (0.089 mL, 0.71 mmol) at room temperature. The reaction mixture was refluxed for 6.5 hours with a Dean-Stark Trap to remove water before being allowed to cool down and concentrated. The residue was chromatographed (silica, hexanes-EtOAc with 1% Et₃N) to give less polar product A (0.485 g, 16%) as a yellow oil, ESIMS m/z=434.10 [M+H]⁺; followed closely by more polar product B (0.230 g, 7.5%) as a yellow oil, ESI MS m/z=434.24 [M+H]⁺.

Step 62e. To a solution of the product A from step 62d (0.485 g, 1.05610.35 mmol) in THF (6 mL) was added 1N HCl (2 mL) at room temperature. The solution was stirred at room temperature for 1 hour and then at 40° C. for 1.5 hours before THF was evaporated off. The remaining aqueous residue was extracted with ether. The aqueous layer was basicified with K₂CO₃ before being extracted with CH₂Cl₂. The combined organics were dried (Na₂SO₄) and evaporated. The residue was chromatographed (silica, hexanes-EtOAc with 1% Et₃N) to give the desired compound (0.244 g, 77%) as a colorless oil. ESIMS m/a=284.06 [M+H]⁺; [α]_{D}²⁰++37.4 (CH₂Cl₂, c 12.2).

Step 62f. The crude desired compound was prepared from the compound from step 62e (80.0 mg, 0.282 mmol) and pyridine-2-carboxaldehyde (36.3 mg, 0.339 mmol) following a similar procedure to that described in step 60d.

Step 62g. The desired compound (0.103 g, 97% over 2 steps) as a colorless oil was prepared from the compound from step 62f (0.282 mmol at most) following a similar procedure to that described in step 60e. ESIMS m/z=375.09 [M+H]⁺.

Step 62h. The desired compound was prepared from the compound of step 62e following similar procedures described in steps 60d and 60e. ESIMS m/z=381.19 [M+H]⁺.

Step 62i. The desired compound (68.3 mg, 76%) as a colorless oil was prepared from the compound from step 62g (53.0 mg, 0.142 mmol) following a similar procedure to that described in step 60f. ESIMS m/z=630.95, 632.95 [M+H]⁺.

Step 62j. The desired compound (61.0 mg, 98%) as a white foam was prepared from the compound from step 62i (68.3 mg, 0.108 mmol) following a similar procedure to that described in step 60g. ESIMS m/z=575.13, 577.13 [M+H]

Step 62k A solution of the compound from step 62j (43.0 mg, 0.074 mmol) and CDI (36.3 mg, 0.224 mmol) in acetonitrile (1 mL) was stirred at room temperature for 1 h. 2-Fluoro-benzenesulfonamide (52.4 mg, 0.299 mmol) was added, followed by DBU (33.0 μL, 0.224 mmol). The mixture was heated at 100° C. for 15 hours before being allowed to cool down and quenched with 1 N HCl solution. The mixture was extracted with EtOAc. The organics were washed (brine), dried (Na₂SO₄), and evaporated. The residue was chromatographed (silica, hexanes-EtOAc) to give the desired compound (9.0 mg, 16%) as a slightly yellow foam. ESIMS m/z=732.00, 734.00 [M+H]⁺.

### Example 63

### Compound of Formula (I), wherein M=Me, Q=3-bromo-4-tert-butylphenyl, Z=benzyl, W═H, Y−—CH₂CH₂CH₇OMe, J=1H-pyrazol-1-ylmethyl

Step 63a. To a solution of the compound from step 62e (65.0 mg, 0.229 mmol) and benzaldehyde (29.2 mg, 0.275 mmol) in CH₂Cl₂ (2 mL) was added MgSO₄ (0.138 g, 1.147 mmol) at room temperature. The suspension was stirred at room temperature over the weekend before being filtered through a short pad of Celite. The filtrate was concentrated to affore the desired compound as a colorless oil, which was used directly for next step.

Step 63b. The desired compound (70.0 mg, 82% over 2 steps) as a colorless oil was prepared from the compound from step 63a (0.229 mmol at most) following a similar procedure to that described in step 60e. ESIMS m/z=374.26 [M+H]⁺.

Step 63c. The desired compound (80.0 mg, 70%) as a colorless oil was prepared from the compound from step 63b (70.0 mg, 0.187 mmol) following a similar procedure to that described in step 60f. ESIMS m/z=630.95, 632.95 [M+H]⁺.

Step 63d. The desired compound (19.0 mg) as a white foam was prepared from the compound from step 63c (80.0 mg, 0.131 mmol) following a similar procedure to that described in step 60g. ESIMS m/z=556.19, 558.19 [M+H]⁺.

Step 63e. The title compound is prepared from the compound of step 63d following a procedure similar to that described in step 1i.

### Example 64

### Compound of Formula (I), wherein M=2-thiophenyl, Q=4-tert-butyl-3-methoxyphenyl, Z=thiazol-2-ylmethyl, W═H, Y=—CH₂CH₂CH₂OMe, J=1H-pyrazol-1-ylmethyl

The title compound was prepared from the compound of step 2c following a procedure similar to that described in step 2d. ESIMS m/z=660.05 [M+H]⁺.

### Example 65

### Compound of Formula (I), wherein M=phenyl, Q=3-bromo-4-tert-butylphenyl, Z=thiazol-2-ylmethyl, W═H, Y=—CH₂CH₂CN, J=1H-pyrazol-1-ylmethyl

Step 65a. To a solution of 4-tert-butylbenzoic acid (10.0 g, 56.1 mmol) in TFA (33 mL) under N₂ were added NBS (12.0 g, 67.3 mmol) and concentrated H₂SO₄ (2 mL) sequentially. The mixture was heated up to 40° C. under N₂ for 18 hours. The reaction was cooled down and diluted with CH₂Cl₂. The organics were washed with water, saturated NaHCO₃, brine, dried over sodium sulfate and evaporated to afford the desired crude compound as an off-white solid (11.2 g, 78%), which was recrystallized from ethyl acetate.

Step 65b. A solution of the compound from step 65a (500 mg, 1.95 mmol) in SOCl₂ (6 mL) was refluxed for 2 hours. It was cooled down and the volatiles were evaporated to afford the desired crude compound as a light yellow oil which was directly used in the next step.

Step 65c. A solution of the compound from step 60b (500 mg, 1.39 mmol) in CH₂Cl₂ (4 mL) was treated with the crude compound from step 65b (1.95 mmol at most) in the presence of Et₃N (0.60 mL, 4.16 mmol) at room temperature for 2 days before being quenched with saturated NaHCO₃ and partitioned between CH₂Cl₂ and water. The organics were washed with brine, dried over sodium sulfate and evaporated. The residue was chromatographed (silica, hexanes-EtOAc) to give the desired compound (0.561 g, 67%) as a yellow solid. ESIMS m/z=599.87, 601.87 [M+H]⁺.

Step 65d. A solution of the compound from step 65c (0.561 g, 0.934 mmol) in dichloromethane (2 mL) was treated with TFA (10 mL) at room temperature for 3 hours before being evaporated. The residue was chromatographed (silica, CH₂Cl₂-methanol) to give the desired compound (0.376 mg, 74%) as a light yellow solid. ESIMS m/z=543.90, 545.90 [M+H]⁺.

Step 65e. The title compound is prepared from the compound of step 65d following a procedure similar to that described in step 2d.

### Example 66

### Compound of Formula (I), wherein M=phenyl, Q=naphthalen-2-yl, Z=thiazol-2-ylmethyl, W═H, Y=—CH₂CH₂CN, J=1H-pyrazol-1-ylmethyl

Step 66a. The desired compound (68.1 mg, NMR 75% purity) as a light yellow solid was prepared from the compound from step 60b (75.0 mg, 0.208 mmol) and 2-naphthoyl chloride (0.119 g, 0.623 mmol) following a similar procedure to that described in step 65c. ESIMS m/z=515.99 [M+H]⁺.

Step 66b. The desired compound (44.3 mg) as a light yellow solid was prepared from the compound from step 66a (68.1 mg, NMR 75% purity) following a similar procedure to that described in step 65d. ESIMS m/z=460.09 [M+H]⁺.

Step 66c. The title compound is prepared from the compound of step 66b following a procedure similar to that described in step 2d.

### Example 67

### Compound of Formula (I), wherein M=phenyl, Q=4-tert-butyl-3-difluoromethylphenyl, Z=thiazol-2-ylmethyl, W═H, Y=—CH₂CH₂CN, J=1H-pyrazol-1-ylmethyl

Step 67a. To a solution of the crude compound from step 65a (3.83 g, 14.9 mmol) in DMF (50 mL) were added t-BuOH (2.84 mL, 29.8 mmol), CDI (3.62 g, 22.3 mmol) and DBU (2.67 mL, 17.9 mmol) sequentially. The mixture was heated to 40° C. under N₂ for 12 hours. The mixture was cooled down and diluted with EtOAc. The organics were washed with brine, dried over sodium sulfate and evaporated. The residue was chromatographed (silica, hexanes-EtOAc) to give the desired compound (4.47 g, 96%) as a colorless oil.

Step 67b. To a solution of the compound from step 67a (1.50 g, 4.79 mmol) in THF (20 mL) at −78° C. was added nBuLi (3.60 mL, 1.6 M in hexane, 5.75 mmol) slowly. The mixture was kept at −78° C. for 1 hour before charging anhydrous DMF (0.73 mL, 9.58 mmol). The reaction was kept at −78° C. for another 2 hours before being quenched with saturated NH₄Cl. The mixture was warmed up to room temperature and partitioned between EtOAc and water. The organics were washed with brine, dried over sodium sulfate and evaporated. The residue was chromatographed (silica, hexanes-EtOAc) to give the desired compound (0.594 g, 48%) as a white solid.

Step 67c. To a solution of the compound from step 67b (0.300 g, 1.15 mmol) in CH₂Cl₂ (12 mL) at −78° C. was added (dimethylamino)sulfur triflouride (DAST, 0.60 mL, 4.58 mmol) slowly. The mixture was gradually warmed up to room temperature and then heated to reflux for 2 hours. The reaction was quenched by saturated NaHCO₃ and the mixture was partitioned between EtOAc and water. The organics were washed with brine, dried over sodium sulfate and evaporated. The residue was chromatographed (silica, hexanes-EtOAc) to give the desired compound (0.266 g, 82%) as a colorless oil.

Step 67d. The desired compound (0.215 g, 100%) as an off-white solid was prepared from the compound from step 67c (266 mg, 0.937 mmol) following a similar procedure to that described in step 65d.

Step 67e. The crude desired compound (0.421 mmol at most) as a light yellow oil was prepared from the compound from step 67d (96.0 mg, 0.421 mmol) following a similar procedure to that described in step 65b.

Step 67f. The desired compound (0.117 g, 62%) as a light yellow solid was prepared from the compound from step 60b (125 mg, 0.346 mmol) and the crude compound from step 67e (0.421 mmol at most) following a similar procedure to that described in step 65c. ESIMS m/z=572.45 [M+H]⁺.

Step 67g. The desired compound (70.0 mg, 67%) as a light yellow solid was prepared from the compound from step 67f (0.117 g, 0.205 mmol) following a similar procedure to that described in step 65d. ESIMS m/z=516.31 [M+H]⁺.

Step 67h. The title compound is prepared from the compound of step 67g following a procedure similar to that described in step 2d.

### Example 68

### Compound of Formula (I), wherein M=phenyl, Q=4-tert-butyl-3-(1,1-difluoroethyl)phenyl, Z=thiazol-2-ylmethyl, W═H, Y=—CH₂CH₂CN, J=1H-pyrazol-1-ylmethyl

Step 68a. To a solution of CBr₄ (0.709 g, 2.14 mmol) in CH₂Cl₂ (8 mL) at 0° C. was added PPh₃ (1.121 g, 4.28 mmol). The resultant mixture was kept at 0° C. for 30 minutes before charging the compound from step 67b (0.280 g, 1.07 mmol) in CH₂Cl₂ (6 mL). The reaction was then kept at 0° C. for 30 minutes before being quenched by saturated NaHCO₃. The mixture was partitioned between EtOAc and water, and the organics were washed with brine, dried over sodium sulfate and evaporated. The residue was chromatographed (silica, hexanes-EtOAc) to give the desired compound (0.338 g, 76%) as a colorless oil.

Step 68b. To a solution of the compound from step 68a (0.337 g, 0.806 mmol) in THF at −78° C. was added nBuLi (1.26 mL, 1.6 M in hexane, 2.02 mmol). The reaction was kept at −78° C. for 2 hours before being quenched with saturated NH₄Cl. The mixture was warmed up to room temperature and partitioned between EtOAc and water. The organics were washed with brine, dried over sodium sulfate and evaporated. The residue was chromatographed (silica, hexanes-EtOAc) to give the desired compound (0.163 g, 79%) as a white solid.

Step 68c. To a solution of HF (˜70% in Py, 10 mL) at 0° C. in a plastic bottle was added the compound from step 68b (0.163 g, 0.632 mmol) slowly. The resultant mixture was warmed up to room temperature and kept stirring for 3 days before being poured into iced water. The mixture was partitioned between CH₂Cl₂ and water, and the organics were washed with brine, dried over sodium sulfate and evaporated. The residue was chromatographed (silica, CH₂Cl₂-MeOH) to give the desired compound (0.120 g, NMR 73% purity) as a white solid.

Step 68d. The crude desired compound (0.403 mmol at most) as a light yellow oil was prepared from the compound from step 68c (0.120 g, NMR 73% purity) following a similar procedure to that described in step 65b.

Step 68e. The desired compound (95.5 mg) as a light yellow oil was prepared from the compound from step 60b (111 mg, 0.307 mmol) and the crude compound from step 68d (0.403 mmol at most) following a similar procedure to that described in step 65c. ESIMS m/z=586.39 [M+H]⁺.

Step 68f. The desired compound (56.5 mg, 66%) as a white solid was prepared from the compound from step 68e (95.5 mg, 0.162 mmol) following a similar procedure to that described in step 65d. ESIMS m/z=530.34 [M+H]⁺.

Step 68g. The title compound is prepared from the compound of step 68f following a procedure similar to that described in step 2d.

### Example 69

### Compound of Formula (I), wherein M=phenyl, Q=4-tert-butyl-3-difluoromethoxyphenyl, Z=thiazol-2-ylmethyl, W═H, Y=—CH₂CH₇CN, J=1H-pyrazol-1-ylmethyl

Step 69a. A mixture of methyl 4-tert-bytulbenzoate (20.0 g, 104 mmol) in concentrated H₂SO₄ (40 mL) was charged with a mixed concentrated H₂SO₄ and concentrated HNO₃ (1:1, 40 mL) via a dropping funnel in an ice-water bath over 20 minutes. It was stirred at 0° C. for 2 hours before pouring into crashed ice (˜400 g). After being stirred for 0.5 hour, it was extracted with ethyl acetate. The organics were washed with water, saturated NaHCO₃, water, brine; dried over sodium sulfate and evaporated to give the crude desired compound as a brownish oil (26.8 g).

Step 69b. A mixture of the crude compound from step 69a (104 mmol at most), ammonium formate (31.5 g) and Pd/C (10%, 2.0 g) in methanol (200 mL) was stirred at room temperature for 15 hours before filtration through Celite. The filtrate was evaporated. The residue was chromatographed (silica, hexanes-EtOAc) to give the desired compound (19.1 g). ESIMS m/z=208.14[M+H]⁺.

Step 69c. A mixture of the compound from step 69b (3.38 g) in acetic acid (35 mL) and water (15 mL) was treated with sodium nitrite (2.25 g) in water (5 mL) at 0° C. over 10 minutes. Another 0.5 hour later, urea (0.98 g) was charged and the mixture was kept stirring for another 5 minutes. Copper sulfate hydrates (33 mg) was added and the mixture was heated at 60-90° C. for 10 minutes before cooling to room temperature. It was partitioned (ethyl acetate and water). The organics were washed with water, saturated NaHCO₃, brine; dried over sodium sulfate and evaporated to give a reddish sirup, which was chromatographed (silica, hexanes-CH₂Cl₂) to give the desired compound (3.08 g).

Step 69d. A mixture of the compound from step 69c (0.200 g, 0.962 mmol), sodium chlorodifluoroacetate (0.733 g, 4.81 mmol) and Cs₂CO₃ (1.57 g, 4.81 mmol) in DMF (10 mL) was heated to 110° C. for 12 hours before being diluted with EtOAc. The organics were washed with brine, dried over sodium sulfate and evaporated. The residue was chromatographed (silica, hexanes-EtOAc) to give the desired compound (0.116 g, 47%) as a colorless oil.

Step 69e. To a solution of the compound from step 69d (0.116 g, 0.450 mmol) in MeOH (5 mL) was added 50% aqueous NaOH (1.8 mL). It was refluxed for 2 hours before being acidified to pH 3. The mixture was partitioned between CH₂Cl₂ and water, and the organics were washed with brine, dried over sodium sulfate and evaporated. The residue was chromatographed (silica, CH₂Cl₂-MeOH) to give the desired compound (99.6 mg, 91%) as a white solid.

Step 69f. The crude desired compound (0.123 mmol at most) as a light yellow oil was prepared from the compound from step 69e (30.0 mg, 0.123 mmol) following a similar procedure to that described in step 65b.

Step 69g. The desired compound (44.5 mg, 68%) as a colorless oil was prepared from the compound from step 60b (40.0 mg, 0.111 mmol) and the crude compound from step 69f (0.123 mmol at most) following a similar procedure to that described in step 65c. ESIMS m/z=588.32 [M+H]⁺.

Step 69h. The desired compound (33.9 mg, 84%) as a light yellow oil was prepared from the compound from step 69g (44.5 mg, 75.8 μmol) following a similar procedure to that described in step 65d. ESIMS m/z=532.30 [M+H]⁺.

Step 69i. The title compound is prepared from the compound of step 69h following a procedure similar to that described in step 2d.

### Example 70

### Compound of Formula (I), wherein M=phenyl, Q=6-bromo-4-tert-butyl-3-methoxyphenyl, Z=thiazol-2-ylmethyl, W═H, Y=—CH₂CH₇CN, J=1H-pyrazol-1-ylmethyl

Step 70a. To a solution of the compound from step 69c (0.920 g, 4.42 mmol) in THF (40 mL) at 0° C. was added NaH (0.265 g, 60% in mineral oil, 6.63 mmol). The mixture was warmed up to room temperature for 1 hour before charging CS₂ (0.53 mL, 8.85 mmol). After 1 hour at room temperature, MeI (2.75 mL, 44.2 mmol) was charged, and the reaction was stirred for 14 hours before being quenched with saturated NH₄Cl. The mixture was partitioned between EtOAc and water, and the organics were washed with brine, dried over sodium sulfate and evaporated. The residue was chromatographed (silica, hexanes-EtOAc) to give the desired compound (0.515 g, NMR 67% purity) as a light yellow oil.

Step 70b. To a solution of 1,3-dibromo-5,5-dimethylhydantoin (0.595 g, 2.08 mmol) in CH₂Cl₂ (3 mL) at −78° C. was added HF (˜70% in Py, 0.70 mL, 27.8 mmol) slowly. It was warmed up to room temperature for 5 minutes before being cooled back to −78° C. and the compound from step 70a (0.595 g, NMR 67% purity) in CH₂Cl₂ (3 mL) was charged. The mixture was gradually warmed up to 0° C. for 30 minutes and saturated NaHCO₃ was charged. It was partitioned between EtOAc and water. The organics were washed with brine, dried over sodium sulfate and evaporated. The residue was chromatographed (silica, hexanes-EtOAc) to give the desired compound (0.491 g) as a white needle crystal.

Step 70c. The desired compound (72.6 mg, 76%) as a white solid was prepared from the compound from step 70b (0.100 g, 0.332 mmol) following a similar procedure to that described in step 69e.

Step 70d. The crude desired compound (0.25 mmol at most) as a light yellow oil was prepared from the compound from step 70c (72.6 mg, 0.25 mmol) following a similar procedure to that described in step 65b.

Step 70e. The desired compound (69.6 mg, 50%) as a colorless oil was prepared from the compound from step 60b (80.0 mg, 0.222 mmol) and the crude compound from step 70d (0.253 mmol at most) following a similar procedure to that described in step 65c.

ESIMS m/z=630.18, 632.18 [M+H]⁺.

Step 70f. The desired compound (54.2 mg, 85%) as a light yellow oil was prepared from the compound from step 70e (69.6 mg, 0.110 mmol) following a similar procedure to that described in step 65d. ESIMS m/z=574.21, 576.21 [M+H]⁺.

Step 70g. The title compound is prepared from the compound of step 70f following a procedure similar to that described in step 2d.

### Example 71

### Compound of Formula (I), wherein M=2-fluorophenyl, Q=4-tent-butyl-3-fluorophenyl, Z=thiazol-2-ylmethyl, W═H, Y=—CH₂CH₇CH₇OMe, J=1H-pyrazol-1-ylmethyl

Step 71a. To a solution of the compound from step 67a (0.300 g, 0.958 mmol) in THF (6 mL) at −78° C. was added nBuLi (0.90 mL, 1.6 M in hexane, 1.44 mmol) slowly. The reaction was kept stirring at −78° C. for 30 minutes before the slow addition of N-fluoro-N-(phenylsulfonyl)benzenesulfonamide (0.756 g, 2.40 mmol) in THF (3 mL). It was gradually warmed up to 0° C. and was quenched with saturated NH₄Cl. The mixture was partitioned between EtOAc and water and the organics were washed with brine, dried over sodium sulfate and evaporated. The residue was chromatographed (silica, hexanes-EtOAc) to give the crude product (0.155 g, NMR 57% purity) as a light yellow oil. The HPLC separation (C₁₈, acetonitrile-20 mM NH₄HCO₃ in water) afford the pure desired compound (55.0 mg, 23%) as a white solid.

Step 71b. The desired compound (34.5 mg, 81%) as a white solid was prepared from the compound from step 71a (55.0 mg, 0.218 mmol) following a similar procedure to that described in step 65d.

Step 71c. The crude desired compound (0.176 mmol at most) as a light yellow oil was prepared from the compound from step 71b (34.5 mg, 0.176 mmol) following a similar procedure to that described in step 65b.

Step 71d. The desired compound (44.8 mg, 46%) as a white solid was prepared from the compound from step 62b (67.0 mg, 0.176 mmol) and the crude compound from step 71c (0.176 mmol at most) following a similar procedure to that described in step 65c. ESIMS m/z=559.37 [M+H]⁺.

Step 71e. The desired compound (18.4 mg, 46%) as a light yellow solid was prepared from the compound from step 71d (44.8 mg, 80.2 μmol) following a similar procedure to that described in step 65d. ESIMS m/z=503.30 [M+H]⁺.

Step 71f. The title compound is prepared from the compound of step 71e following a procedure similar to that described in step 2d.

### Example 72

### Compound of Formula (I), wherein M=2-fluorophenyl, Q=4-tert-butyl-3-chlorophenyl, Z=thiazol-2-ylmethyl, W═H, Y=—CH₂CH₂CH₂OMe, J=1H-pyrazol-1-ylmethyl

Step 72a. To a solution of the compound from step 67a (0.300 g, 0.958 mmol) in THF (6 mL) at −78° C. was added nBuLi (0.90 mL, 1.6 M in hexane, 1.44 mmol) slowly. It was stirred at −78° C. for 30 minutes before the slow addition of perchloroethane (0.567 g, 2.40 mmol) in THF (3 mL). The mixture was gradually warmed up to room temperature for 3 hours before being quenched with saturated NH₄Cl. It was partitioned between EtOAc and water and the organics were washed with brine, dried over sodium sulfate and evaporated. The residue was chromatographed (silica, hexanes-EtOAc) to give the desired compound (0.193 g, NMR 75% purity) as a colorless oil.

Step 72b. The desired compound (0.172 g, NMR 75% purity) as a white solid was prepared from the compound from step 72a (0.193 g, NMR 75% purity) following a similar procedure to that described in step 65d.

Step 72c. The crude desired compound (0.718 mmol at most) as a light yellow oil was prepared from the compound from step 72b (0.172 g, NMR 75% purity) following a similar procedure to that described in step 65b.

Step 72d. The desired compound (0.114 g) as a light yellow solid was prepared from the compound from step 62b (0.210 g, 0.552 mmol) and the crude compound from step 72c (0.718 mmol at most) following a similar procedure to that described in step 65c. ESIMS m/z=575.36, 577.36 [M+H]⁺.

Step 72e. The desired compound (74.0 mg, 72%) as a light yellow solid was prepared from the compound from step 72d (0.114 g, 0.199 mmol) following a similar procedure to that described in step 65d. ESIMS m/z=519.28, 521.28 [M+H]⁺.

Step 72f. The title compound (5.3 mg, 41%) as a white solid was prepared from the compound of step 72e (10.0 mg, 19.2 μmol) and 2-fluorobenzenesulfonamide (13.5 mg, 76.8 μmol) following a procedure similar to that described in step 2d. ESIMS m/z=676.11, 678.11 [M+H]⁺.

### Example 73

### Compound of Formula (I), wherein M=2-fluorophenyl, Q=4-tert-butyl-6-fluoro-3-methoxyphenyl, Z=thiazol-2-ylmethyl, W═H, Y=—CH₂CH₂CH₂OMe, J=1H-pyrazol-1-ylmethyl

Step 73a. To a solution of Et₃N (6.35 mL, 44.1 mmol) in MeOH (100 mL) at 0° C. was added the compound from step 1f (5.00 g, 22.1 mmol) slowly. It was warmed up to room temperature and stirred for 12 hours. The volatiles were evaporated to afford the desired compound (4.88 g, 100%) as a light yellow oil.

Step 73b. To a solution of the compound from step 73a (4.86 g, 22.0 mmol) in trifluoroacetic anhydride (50 mL) was added silver nitrate (4.86 g, 28.6 mmol) in portions slowly. It was stirred for 5 hours before being poured into ice-water. The mixture were partitioned between EtOAc and water. The organics were washed with saturated NaHCO₃, brine, dried over sodium sulfate and evaporated. The residue was chromatographed (silica, hexanes-EtOAc) to give the desired compound (1.977 g) as a light yellow solid.

Step 73c. To a solution of the compound from step 73b (1.50 g, 5.67 mmol) in MeOH (60 mL) was added Pd/C (10%, 0.15 g) and ammonium formate (2.13 g, 33.7 mmol). It was stirred for 16 hours before filtration. The filtrate was evaporated to dryness. The residue were partitioned between EtOAc and water, and the organics were washed with saturated NaHCO₃, brine, dried over sodium sulfate and evaporated. The residue was chromatographed (silica, hexanes-EtOAc) to give the desired compound (0.870 g, 65%) as a white solid.

Step 73d. To a solution of boron trifluoride etherate (0.41 mL, 4.06 mmol) in 1,2-dimethoxyethane (DME, 15 mL) at −10° C. was added the compound from step 73c (0.770 g, 3.25 mmol) in DME (21 mL) dropwise. The reaction was stirred at this temperature for 30 minutes before the slow addition of tent-butyl nitrite (0.45 mL, 90%, 3.41 mmol) in DME (15 mL). The mixture was warmed up to 0° C. and stirred for 1.5 hours before being evaporated to dryness. The residue was dissolved into chlorobenzene (40 mL) and was heated up to 135° C. for 1.5 hours. The reaction was cooled down and the volatiles were evaporated. The residue was chromatographed (silica, hexanes-EtOAc) to give the desired compound (0.232 g) as a yellow oil.

Step 73e. The desired compound (0.230 g, 91%) as a white solid was prepared from the compound from step 73d (0.270 g, 1.13 mmol) following a similar procedure to that described in step 69e.

Step 73f. The crude desired compound (1.02 mmol at most) as a light yellow oil was prepared from the compound from step 73e (0.230 g, 1.02 mmol) following a similar procedure to that described in step 65b.

Step 73g. The desired compound (0.323 g, 60%) as a light yellow oil was prepared from the compound from step 62b (0.350 g, 0.921 mmol) and the crude compound from step 73f (1.02 mmol at most) following a similar procedure to that described in step 65c. ESIMS m/z=589.13 [M+H]⁺.

Step 73h. The desired compound (20.4 mg, 81%) as a light yellow oil was prepared from the compound from step 73g (28.0 mg, 47.6 μmol) following a similar procedure to that described in step 65d. ESIMS m/z=533.09 [M+H]⁺.

Step 73i. The title compound (1.8 mg) as a white solid was prepared from the compound of step 73h (10.0 mg, 18.7 μmol) and 2-fluorobenzenesulfonamide (16.4 mg) following a procedure similar to that described in step 2d. ESIMS m/z=690.26 [M+H]⁺.

### Example 74

### Compound of Formula (I), wherein M=2-fluorophenyl, Q=7-tert-butyl-benzoxazol-4-yl, Z=thiazol-2-ylmethyl, W═H, Y=—CH₂CH₇CH₇OMe, J=1H-pyrazol-1-ylmethyl

Step 74a. The desired compound (3.474 g, NMR 55% purity) was obtained as a side product in step 73b.

Step 74b. To a solution of the compound from step 74a (3.474 g) in MeOH (60 mL) was added Pd/C (10%, 0.35 g) and ammonium formate (4.92 g, 78.1 mmol) sequentially. The mixture was stirred for 16 hours before filtration. The filtrate was evaporated to dryness. The residue were partitioned between EtOAc and water, and the organics were washed with sat. NaHCO₃, brine, dried over sodium sulfate and evaporated. The residue was chromatographed (silica, hexanes-EtOAc) to give the desired compound (0.281 g) as a yellow solid.

Step 74c. The compound from step 74b (0.130 g, 0.583 mmol) was dissolved into 4N HCl in dioxane (3 mL), and was then evaporated to dryness. The residue was then dissolved into CH₂Cl₂ (6 mL) and was cooled down to −78° C. Boron tribromide (2.90 mL, 1 M in CH₂Cl₂, 2.91 mmol) was added into the reaction, and the resultant mixture was slowly warmed up to room temperature. The reaction was kept stirring for 10 hours before being quenched with saturated NaHCO₃. The mixture was partitioned between CH₂Cl₂ and water, and the organics were washed with saturated NaHCO₃, brine, dried over sodium sulfate and evaporated. The residue was chromatographed (silica, CH₂Cl₂-MeOH) to give the desired compound (71.0 mg, 58%) as a yellow solid.

Step 74d. To a solution of the compound from step 74c (71.0 mg, 0.340 mmol) in DMF (3 mL) were added trimethyl orthoformate (3 mL) and TsOH (13.0 mg, 67.9 μmol). The resultant mixture was kept stirring for 24 hours before being diluted with water (50 mL). The solution was lyophilized to afford the desired compound (66.0 mg, 84%) as a light yellow solid.

Step 74e. The crude desired compound (0.301 mmol at most) as a light yellow oil was prepared from the compound from step 74d (66.0 mg, 0.301 mmol) following a similar procedure to that described in step 65b.

Step 74f. The desired compound (75.1 mg, NMR 67% purity) as a colorless oil was prepared from the compound from step 62b (75.0 mg, 0.201 mmol) and the crude compound from step 74e (0.301 mmol at most) following a similar procedure to that described in step 65c. ESIMS m/z=582.33 [M+H]⁺.

Step 74g. The desired compound (45.2 mg) as a white solid was prepared from the compound from step 74f (75.1 mg, NMR 67% purity) following a similar procedure to that described in step 65d. ESIMS m/z=526.22 [M+H]⁺.

Step 74h. The title compound is prepared from the compound of step 74g following a procedure similar to that described in step 2d.

### Example 75

### Compound of Formula (I), wherein M=2-fluorophenyl, Q=4-tert-butyl-3-ethoxylphenyl, Z=thiazol-2-ylmethyl, W═H, Y=—CH₂CH₂CH₂OMe, J=1H-pyrazol-1-ylmethyl

Step 75a. A solution of the compound from step 69c (142 mg, 0.68 mmol) in DMF (5 mL) was treated with NaH (60% in mineral oil, 50 mg, 1.25 mmol) at room temperature for 20 minutes before charging ethyl iodide (0.20 mL, 2.5 mmol). It was quenched with acetic acid after 3 hours, partitioned (hexanes-ethyl acetate). The aqueous was extracted with the same solvents. The combined organics were washed with saturated NaHCO₃, water and brine, dried over sodium sulfate and evaporated. The residue was chromatographed (silica, hexanes-EtOAc) to give the desired compound (151 mg) as a mixture of methyl and ethyl ester.

Step 75b. A solution of the compound from step 15a (151 mg) in methanol (5 mL) was treated with aqueous NaOH (50%, 0.17 mL) under reflux for 3 hours before cooling and acidifying to pH˜3. It was evaporated, dried in vacuo, and chromatographed (silica, EtOAc) to give the desired compound (146 mg).

Step 75c. The crude desired compound (0.189 mmol at most) as a light yellow oil was prepared from the compound from step 75b (42.0 mg, 0.189 mmol) following a similar procedure to that described in step 65b.

Step 75d. The desired compound (51.9 mg, 61%) as a colorless oil was prepared from the compound from step 62b (55.0 mg, 0.145 mmol) and the crude compound from step 75c (0.189 mmol at most) following a similar procedure to that described in step 65c. ESIMS m/z=585.19 [M+H]⁺.

Step 75e. The desired compound (36.3 mg, 77%) as a colorless oil was prepared from the compound from step 75d (51.9 mg, 88.8 μmol) following a similar procedure to that described in step 65d. ESIMS m/z=529.13 [M+H]⁺.

Step 75f. The title compound is prepared from the compound of step 75e following a procedure similar to that described in step 2d.

### Example 76

### Compound of Formula (Ia), wherein M=2-fluorophenyl, Q=4-tert-butyl-6-fluoro-3-methoxyphenyl, Z=thiazol-2-ylmethyl, W═H, Y=—CH₂CH₂CH₂OMe, J=1H-pyrazol-1-ylmethyl

Step 76a. The racemic compound from step 73g (112 mg, 0.190 mmol) was separated by chiral HPLC separation (Lux Cellulose-1, isopropanol-hexanes). The desired compound was obtained (27.4 mg) as the collection of the first peak (retention time 14.2 minutes).

Step 76b. The desired compound (22.0 mg, 92%) as a colorless oil was prepared from the compound from step 76a (26.3 mg, 44.2 μmol) following a similar procedure to that described in step 65d. ESIMS m/z=533.18 [M+H]⁺.

Step 76c. The title compound (7.5 mg) as a white solid was prepared from the compound of step 76b (20.0 mg, 37.5 μmmol) and 2-fluorobenzenesulfonamide (32.8 mg, 0.187 mmol) following a procedure similar to that described in step 2d. ESIMS m/z=690.28 [M+H]⁺.

### Example 77

### The Opposite Enantiomer of Compound of Formula (Ia), wherein M=2-fluorophenyl, Q=4-tent-butyl-6-fluoro-3-methoxyphenyl, Z=thiazol-2-ylmethyl, W═H, Y=—CH₂CH₂CH₂OMe, J=1H-pyrazol-1-ylmethyl

Step 77a. The desired compound was obtained (21.2 mg) as the collection of the second peak (retention time 18.3 minutes) in step 76a during chiral HPLC separation.

Step 77b. The desired compound (16.6 mg, 89%) as a colorless oil was prepared from the compound from step 77a (20.6 mg, 35.0 μmol) following a similar procedure to that described in step 65d. ESIMS m/z=533.18 [M+H]⁺.

Step 77c. The title compound (5.1 mg) as a white solid was prepared from the compound of step 77b (14.6 mg, 27.4 μmol) and 2-fluorobenzenesulfonamide (19.2 mg, 0.110 mmol) following a procedure similar to that described in step 2d. ESIMS m/z=690.11 [M+H]⁺.

### Example 78

### Compound of Formula (Ia), wherein M=2-fluorophenyl, Q=3-bromo-4-tert-butyl-6-fluorophenyl, Z=thiazol-2-ylmethyl, W═H, Y=—CH₂CH₂CH₂OMe, J=1H-pyrazol-1-ylmethyl

Step 78a. A solution of the crude compound from step 65a (2.00 g, 7.78 mmol) in benzene (35 mL) and MeOH (4 mL) was treated with methanesulfonic acid (1.12 g, 11.7 mmol) under reflux with a Dean-Stark apparatus for 12 hours. It was cooled down and partitioned between EtOAc and water. The organics were washed with saturated NaHCO₃, brine, dried over sodium sulfate and evaporated to give the crude desired compound (2.17 g, 100%) as a light yellow oil.

Step 78b. The desired compound (1.96 g, 80%) was prepared from the compound of step 78a (2.17 g, 7.78 mmol) following a procedure similar to that described in step 69a.

Step 78c. To a solution of the compound from step 78b (0.550 g, 1.74 mmol) in EtOH (8 mL) and EtOAc (8 mL) was added stannous chloride hydrate (SnCl₂.2H₂O, 1.79 g, 7.91 mmol). The reaction was heated up to 70° C. for 30 minutes before being evaporated to dryness. The residue was partitioned between EtOAc and water, and the organics were washed with saturated NaHCO₃, brine, dried over sodium sulfate and evaporated. The residue was chromatographed (silica, hexanes-EtOAc) to give the desired compound (0.475 g, 96%) as a colorless oil.

Step 78d. The desired compound (0.322 g) was prepared from the compound of step 78c (0.475 g, 1.66 mmol) following a procedure similar to that described in step 73d.

Step 78e. The desired compound (0.301 g, 98%) as a white solid was prepared from the compound from step 78d (0.301 g, 1.11 mmol) following a similar procedure to that described in step 69e.

Step 78f. The crude desired compound (0.174 mmol at most) as a light yellow oil was prepared from the compound from step 78e (0.301 g, 1.09 mmol) following a similar procedure to that described in step 65b.

Step 78g. The desired compound (45.7 mg, 53%) as a colorless oil was prepared from the compound from step 62h (51.0 mg, 0.134 mmol) and the crude compound from step 78f (0.174 mmol at most) following a similar procedure to that described in step 65c. ESIMS m/z=637.10, 639.10 [M+H]⁺.

Step 78h. The desired compound (36.5 mg, 88%) as a colorless oil was prepared from the compound from step 78g (45.7 mg, 71.7 μmol) following a similar procedure to that described in step 65d. ESIMS m/z=581.09, 583.09 [M+H]⁺.

Step 78i. The title compound (13.4 mg) as a white solid was prepared from the compound of step 78h (34.0 mg, 58.5 μmol) and 2-fluorobenzenesulfonamide (25.6 mg, 0.146 mmol) following a procedure similar to that described in step 2d. ESIMS m/z=738.04, 740.03 [M+H]⁺.

### Example 79

### Compound of Formula (Ia), wherein M=2-fluorophenyl, Q=3-bromo-4-tert-butyl-2-fluorophenyl, Z=thiazol-2-ylmethyl, W═H, Y=—CH₂CH₂CH₂OMe, J=1H-pyrazol-1-ylmethyl

Step 79a. The desired compound (0.355 g, 14%) as a colorless oil was obtained as a side product in step 78b.

Step 79b. To a solution of the compound from step 79a (0.355 g, 1.12 mmol) in EtOH (8 mL) and EtOAc (8 mL) was added SnCl₂.2H₂O (2.03 g, 8.99 mmol). It was heated at 70° C. for 20 hours before being evaporated to dryness. The residue was partitioned between EtOAc and water, and the organics were washed with saturated NaHCO₃, brine, dried over sodium sulfate and evaporated. The residue was chromatographed (silica, hexanes-EtOAc) to give the desired compound (80.5 mg, 25%) as a colorless oil.

Step 79c. To a solution of BF₃.Et₂O (38.0 μL, 0.378 mmol) in DME (4 mL) at −10° C. was added the compound from step 79b (80.5 mg, 0.280 mmol) in DME (6 mL) dropwise. The reaction was kept stirring at this temperature for 30 minutes before the slow addition of tent-butyl nitrite (41.0 μL, 90%, 0.308 mmol) in DME (10 mL). The mixture was warmed up to 0° C. and kept stirring for 1.5 hours before being evaporated to dryness. The residue was dissolved into xylene (10 mL) and was heated up to 135° C. for 30 minutes. The reaction was cooled down and the volatiles were evaporated. The residue was chromatographed (silica, hexanes-EtOAc) to give the desired compound (72.0 mg, NMR 75% purity) as a light yellow oil.

Step 79d. To a solution of the compound from step 79c (72.0 mg, NMR 75% purity) in MeOH (6 mL) was added 50% aqueous NaOH (0.13 mL). The reaction was heated to reflux for 2 hours before being acidified to pH 3. The mixture was partitioned between CH₂Cl₂ and water, and the organics were washed with brine, dried over sodium sulfate and evaporated. The residue was chromatographed (silica, CH₂Cl₂-MeOH) to give the desired compound (60.2 mg, NMR 75% purity) as a white solid.

Step 79e. The crude desired compound (0.220 mmol at most) as a light yellow oil was prepared from the compound from step 79d (60.2 mg, NMR 75% purity) following a similar procedure to that described in step 65b.

Step 79f. The desired compound (29.0 mg) as a colorless oil was prepared from the compound from step 62h (62.0 mg, 0.163 mmol) and the crude compound from step 79e (0.220 mmol at most) following a similar procedure to that described in step 65c; and a portion of the crude was purified by HPLC (C₁₈, acetonitrile-20 mM NH₄HCO₃ in water). ESIMS m/z=637.10, 639.10 [M+H]⁺.

Step 79g. The desired compound (22.0 mg, 83%) as a colorless oil was prepared from the compound from step 79f (29.0 mg, 45.5 μmol) following a similar procedure to that described in step 65d. ESIMS m/z=581.17, 583.17 [M+H]⁺.

Step 79h. The title compound (6.1 mg) as a white solid was prepared from the compound of step 79g (20.0 mg, 34.4 μmol) and 2-fluorobenzenesulfonamide (21.1 mg, 0.120 mmol) following a procedure similar to that described in step 2d. ESIMS m/z=737.99, 739.98 [M+H]⁺.

### Example 80

### The Compound of Formula (Ia), wherein M=2-fluorophenyl, Q=3-bromo-4-tert-butylphenyl, Z=thiazol-2-ylmethyl, W═H, Y=—CH₂CH₂CH₇OMe, J=1H-pyrazol-1-ylmethyl

Step 80a. The desired compound (5.6 mg) as a colorless oil was obtained in step 79f as a contaminant which could be traced back to side product in step 79c. ESIMS m/z=619.11, 621.11 [M+H]⁺.

Step 80b. The desired compound (5.2 mg, 100%) as a colorless oil was prepared from the compound from step 80a (5.6 mg, 9.0 μmol) following a similar procedure to that described in step 65d. ESIMS m/z=563.07, 565.07 [M+H]⁺.

Step 80c. The title compound is prepared from the compound of step 80b following a procedure similar to that described in step 2d.

### Example 81

### Compound of Formula (Ia), wherein M=2-fluorophenyl, Q=4-tert-butyl-3-methoxyphenyl, Z=thiazol-2-ylmethyl, W═H, Y=—CH₂CH₇CH₂OMe, J=1H-pyrazol-1-ylmethyl

The title compound was prepared following procedures similar to that described for the compound of example 76. ESIMS m/z=672.00 [M+H]⁺.

While this invention has been particularly shown and described with references to preferred embodiments thereof, it will be understood by those skilled in the art that various changes in form and details may be made therein without departing from the scope of the invention encompassed by the appended claims.

What is claimed is:

1. A compound represented by Formula (I): 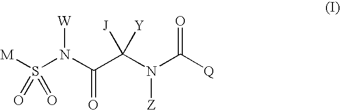
or a pharmaceutically acceptable salt, ester, stereoisomer, tautomer, prodrug, solvate, or combination thereof, wherein:
M is —R₁ or —NR₂R₂ₐ; wherein R₂ and R₂ₐ are each independently hydrogen or —R₁; or R₂ and R₂ₐ taken together with the nitrogen atom to which they are attached form an optionally substituted heterocyclic or optionally substituted heteroaryl group; and R₁ at each occurrence is independently selected from the group consisting of: optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted heterocyclic, optionally substituted aryl and optionally substituted heteroaryl;
W is hydrogen or hydroxy;
Q is an optionally substituted aryl or optionally substituted heteroaryl;
Y is selected from the group consisting of: optionally substituted —C₁-C₈ alkyl, optionally substituted —C₃-C₆ alkenyl or optionally substituted —C₃-C₆ alkynyl each containing 0, 1, 2, or 3 heteroatoms selected from O, S or N;
J is —C₁-C₄ alkyl substituted with —O—C₁-C₄ alkyl, —N(—C₁-C₄ alkyl)₂, optionally substituted heterocyclic, optionally substituted aryl or optionally substituted heteroaryl; and
Z is a —C₁-C₄ alkyl substituted with an optionally substituted heterocyclic, optionally substituted aryl or optionally substituted heteroaryl.

2. A compound of claim 1 wherein W is hydrogen and M is —R₁ or a pharmaceutically acceptable salt, ester, stereoisomer, tautomer, solvate, prodrug, or combination thereof.

3. A compound of claim 1 wherein W is hydrogen and M is —NR₂R₂ₐ or a pharmaceutically acceptable salt, ester, stereoisomer, tautomer, solvate, prodrug, or combination thereof.

4. A compound of claim 1 wherein W is hydrogen and J is a methyl or ethyl substituted with optionally substituted aryl or optionally substituted heteroaryl or a pharmaceutically acceptable salt, ester, stereoisomer, tautomer, solvate, prodrug, or combination thereof.

5. A compound of claim 1 wherein W is hydrogen and J is —C₁-C₄ alkyl substituted with —O—C₁-C₄ alkyl, —N(—C₁-C₄ alkyl)₂ or optionally substituted heterocyclic or a pharmaceutically acceptable salt, ester, stereoisomer, tautomer, solvate, prodrug, or combination thereof.

6. A compound of claim 1 wherein W is hydrogen and Q is a substituted phenyl or substituted pyridyl or a pharmaceutically acceptable salt, ester, stereoisomer, tautomer, solvate, prodrug, or combination thereof.

7. A compound of claim 1 wherein W is hydrogen and Z is —C₁-C₄ alkyl substituted with optionally substituted aryl or optionally substituted heteroaryl or a pharmaceutically acceptable salt, ester, stereoisomer, tautomer, solvate, prodrug, or combination thereof.

8. A compound of claim 1 wherein W is hydrogen and Y is a substituted —C₁-C₄ alkyl containing 0, 1, 2, or 3 heteroatoms selected from O, S or N or a pharmaceutically acceptable salt, ester, stereoisomer, tautomer, solvate, prodrug, or combination thereof.

9. A compound of claim 1 wherein W is hydrogen, J is a methyl substituted with optionally substituted aryl or optionally substituted heteroaryl, Y is a substituted —C₁-C₄ alkyl containing 0, 1, 2, or 3 heteroatoms selected from O, S or N, and Z is a methyl substituted with optionally substituted aryl or optionally substituted heteroaryl, and Q is a substituted phenyl or a pharmaceutically acceptable salt, ester, stereoisomer, tautomer, solvate, prodrug, or combination thereof.

10. A compound according to claim 1 selected from the group consisting of:
Compound of Formula (I), wherein M=Me, Q=4-tent-butyl-3-methoxyphenyl, Z=thiazol-2-ylmethyl, W═H, Y=—CH₂CH═CH₂, J=1H-pyrazol-1-ylmethyl;
Compound of Formula (I), wherein M=Me, Q=4-tent-butyl-3-methoxyphenyl, Z=thiazol-2-ylmethyl, W═H, Y=—CH₂CH₂CH₂OMe, J=1H-pyrazol-1-ylmethyl;
Compound of Formula (I), wherein M=Me, Q=4-tent-butyl-3-methoxyphenyl, Z=thiazol-2-ylmethyl, W═H, Y=—CH₂CH₂CH═NOMe, J=1H-pyrazol-1-ylmethyl;
Compound of Formula (I), wherein M=Me, Q=4-tent-butyl-3-methoxyphenyl, Z=thiazol-2-ylmethyl, W═H, Y=—CH₂CH₂CH₂NMe₂, J=1H-pyrazol-1-ylmethyl;
Compound of Formula (I), wherein M=Me, Q=4-tent-butyl-3-methoxyphenyl, Z=thiazol-2-ylmethyl, W═H, Y=(E)-CH₂CH₂CH═CHCN, J=1H-pyrazol-1-ylmethyl;
Compound of Formula (I), wherein M=Me, Q=4-tent-butyl-3-methoxyphenyl, Z=thiazol-2-ylmethyl, W═H, Y=(Z)-CH₂CH₂CH═CHCN, J=1H-pyrazol-1-ylmethyl;
Compound of Formula (I), wherein M=Me, Q=4-tent-butyl-3-methoxyphenyl, Z=thiazol-2-ylmethyl, W═H, Y=—CH₂CH₂CH₂CH₂CN, J=1H-pyrazol-1-ylmethyl;
Compound of Formula (I), wherein M=Me, Q=4-tent-butyl-3-methoxyphenyl, Z=thiazol-2-ylmethyl, W═H, Y=—CH₂CH₂CH═CF₂, J=1H-pyrazol-1-ylmethyl;
Compound of Formula (I), wherein M=Me, Q=4-tent-butyl-3-methoxyphenyl, Z=thiazol-2-ylmethyl, W═H, Y=—CH₂CHO, J=1H-pyrazol-1-ylmethyl;
Compound of Formula (I), wherein M=Me, Q=4-tent-butyl-3-methoxyphenyl, Z=thiazol-2-ylmethyl, W═H, Y=—CH₂CH₂OMe, J=1H-pyrazol-1-ylmethyl;
Compound of Formula (I), wherein M=Me, Q=4-tent-butyl-3-methoxyphenyl, Z=thiazol-2-ylmethyl, W═H, Y=(E)-CH₂CH═CHCN, J=1H-pyrazol-1-ylmethyl;
Compound of Formula (I), wherein M=Me, Q=4-tent-butyl-3-methoxyphenyl, Z=thiazol-2-ylmethyl, W═H, Y=(Z)-CH₂CH═CHCN, J=1H-pyrazol-1-ylmethyl;
Compound of Formula (I), wherein M=Me, Q=4-tent-butyl-3-methoxyphenyl, Z=thiazol-2-ylmethyl, W═H, Y=(E)-CH₂CH₂CH₂CN, J=1H-pyrazol-1-ylmethyl;
Compound of Formula (I), wherein M=Me, Q=4-tent-butyl-3-methoxyphenyl, Z=thiazol-2-ylmethyl, W═H, Y=—CH₂CH═NOMe, J=1H-pyrazol-1-ylmethyl;
Compound of Formula (I), wherein M=Me, Q=4-tent-butyl-3-methoxyphenyl, Z=thiazol-2-ylmethyl, W═H, Y=—CH₂CH₂NMe₂, J=1H-pyrazol-1-ylmethyl;
Compound of Formula (I), wherein M=Me, Q=4-tent-butyl-3-methoxyphenyl, Z=thiazol-2-ylmethyl, W═H, Y=—CH₂CH═CH₂, J=1H-pyrazol-1-ylmethyl;
Compound of Formula (I), wherein M=Me, Q=4-tent-butyl-3-methoxyphenyl, Z=thiazol-2-ylmethyl, W═H, Y=—CH₂CH═CH₂, J=1H-pyrazol-1-ylmethyl;
Compound of Formula (I), wherein M=-Ph, Q=4-tent-butyl-3-methoxyphenyl, Z=thiazol-2-yl, W═H, Y=—CH₂CH₂CN, J=1H-pyrazol-1-ylmethyl;
Compound of Formula (I), wherein M=-Ph, Q=4-tent-butyl-3-methoxyphenyl, Z=thiazol-2-yl, W═H, Y=—CH₂CN, J=1H-pyrazol-1-ylmethyl;
Compound of Formula (I), wherein M=-Ph, Q=4-tent-butyl-3-methoxyphenyl, Z=thiazol-2-yl, W═H, Y=—CH₂CH₂-(tetrazol-5-yl), J=1H-pyrazol-1-ylmethyl;
Compound of Formula (I), wherein M=-Ph, Q=4-tent-butyl-3-methoxyphenyl, Z=thiazol-2-yl, W═H, Y=—CH₂-(tetrazol-5-yl), J=1H-pyrazol-1-ylmethyl;
Compound of Formula (I), wherein M=—NH₂, Q=4-tent-butyl-3-methoxyphenyl, Z=thiazol-2-ylmethyl, W═H, Y=—CH₂CH₂CH₂OMe, J=1H-pyrazol-1-ylmethyl;
Compound of Formula (I), wherein M=—NHPh, Q=4-tent-butyl-3-methoxyphenyl, Z=thiazol-2-ylmethyl, W═H, Y=—CH₂CH₂CH₂OMe, J=1H-pyrazol-1-ylmethyl;
Compound of Formula (I), wherein M=-NMe₂, Q=4-tent-butyl-3-methoxyphenyl, Z=thiazol-2-ylmethyl, W═H, Y=—CH₂CH₂CH₂OMe, J=1H-pyrazol-1-ylmethyl;
Compound of Formula (I), wherein M=-Ph, Q=4-tent-butyl-3-methoxyphenyl, Z=thiazol-2-ylmethyl, W═OH, Y=—CH₂CH₂CH₂OMe, J=1H-pyrazol-1-ylmethyl;
Compound of Formula (I), wherein M=-Phenyl-fluoro-(p), Q=4-tent-butyl-3-methoxyphenyl, Z=thiazol-2-ylmethyl, W═H, Y=—CH₂CH₂CH₂OMe, J=1H-pyrazol-1-ylmethyl;
Compound of Formula (I), wherein M=-Phenyl-fluoro-(m), Q=4-tent-butyl-3-methoxyphenyl, Z=thiazol-2-ylmethyl, W═H, Y=—CH₂CH₂CH₂OMe, J=1H-pyrazol-1-ylmethyl;
Compound of Formula (I), wherein M=-Phenyl-fluoro-(O), Q=4-tent-butyl-3-methoxyphenyl, Z=thiazol-2-ylmethyl, W═H, Y=—CH₂CH₂CH₂OMe, J=1H-pyrazol-1-ylmethyl;
Compound of Formula (I), wherein M=-2-pyridyl, Q=4-tent-butyl-3-methoxyphenyl, Z=thiazol-2-ylmethyl, W═H, Y=—CH₂CH₂CH₂OMe, J=1H-pyrazol-1-ylmethyl;
Compound of Formula (I), wherein M=-3-pyridyl, Q=4-tent-butyl-3-methoxyphenyl, Z=thiazol-2-ylmethyl, W═H, Y=—CH₂CH₂CH₂OMe, J=1H-pyrazol-1-ylmethyl;
Compound of Formula (I), wherein M=-4-pyridiyl, Q=4-tent-butyl-3-methoxyphenyl, Z=thiazol-2-ylmethyl, W═H, Y=—CH₂CH₂CH₂OMe, J=1H-pyrazol-1-ylmethyl;
Compound of Formula (I), wherein M=-Ph, Q=4-tent-butyl-3-trifluoromethoxyphenyl, Z=thiazol-2-ylmethyl, W═H, Y=—CH₂CH₂CH₂OMe, J=1H-pyrazol-1-ylmethyl;
Compound of Formula (I), wherein M=-Ph, Q=5-tent-butyl-4-methoxypyridin-2-yl, Z=thiazol-2-ylmethyl, W═H, Y=—CH₂CH₂CH₂OMe, J=1H-pyrazol-1-ylmethyl;
Compound of Formula (I), wherein M=-Ph, Q=4-tent-butyl-3-vinylphenyl, Z=thiazol-2-ylmethyl, W═H, Y=—CH₂CH₂CH₂OMe, J=1H-pyrazol-1-ylmethyl;
Compound of Formula (I), wherein M=-Ph, Q=4-tent-butyl-3-bromophenyl, Z=thiazol-2-ylmethyl, W═H, Y=—CH₂CH₂CH₂OMe, J=1H-pyrazol-1-ylmethyl;
Compound of Formula (I), wherein M=-Ph, Q=4-tent-butyl-3-chlorophenyl, Z=thiazol-2-ylmethyl, W═H, Y=—CH₂CH₂CH₂OMe, J=1H-pyrazol-1-ylmethyl;
Compound of Formula (I), wherein M=-Ph, Q=4-tent-butyl-3-fluorophenyl, Z=thiazol-2-ylmethyl, W═H, Y=—CH₂CH₂CH₂OMe, J=1H-pyrazol-1-ylmethyl;
Compound of Formula (I), wherein M=-Ph, Q=4-tent-butyl-5-bromo-2-fluorophenyl, Z=thiazol-2-ylmethyl, W═H, Y=—CH₂CH₂CH₂OMe, J=1H-pyrazol-1-ylmethyl;
Compound of Formula (I), wherein M=-Ph, Q=4-tent-butyl-5-bromo-2-fluorophenyl, Z=(5-methyl-isoxazol-3-yl)methyl, W═H, Y=—CH₂CH₂CH₂OMe, J=1H-pyrazol-1-ylmethyl;
Compound of Formula (I), wherein M=-Ph, Q=4-tent-butyl-5-bromo-2-fluorophenyl, Z=thiazol-2-ylmethyl, W═H, Y=—CH₂CH₂CH₂OMe, J=1,3-thiazol-4-ylmethyl;
Compound of Formula (I), wherein M=-Ph, Q=4-tent-butyl-5-bromo-2-fluorophenyl, Z=thiazol-2-ylmethyl, W═H, Y=—CH₂CH₂CH₂OMe, J=1,3-thiazol-2-ylmethyl;
Compound of Formula (I), wherein M=-Ph, Q=4-tent-butyl-5-bromo-2-fluorophenyl, Z=thiazol-2-ylmethyl, W═H, Y=—CH₂CH₂CH₂OMe, J=isothiazol-3-ylmethyl;
Compound of Formula (I), wherein M=-Ph, Q=4-tent-butyl-5-bromo-2-fluorophenyl, Z=Bn, W═H, Y=—CH₂CH₂CH₂OMe, J=isothiazol-3-ylmethyl;
Compound of Formula (I), wherein M=-Ph, Q=4-tent-butyl-5-bromo-2-fluorophenyl, Z=thiazol-2-ylmethyl, W═H, Y=—CH₂CH₂CH₂OMe, J=Bn;
Compound of Formula (I), wherein M=-Ph, Q=4-tent-butyl-3-methoxyphenyl, Z=thiazol-2-ylmethyl, W═H, Y=propargyl, J=1H-pyrazol-1-ylmethyl;
Compound of Formula (I), wherein M=-Ph, Q=4-tent-butyl-3-methoxyphenyl, Z=thiazol-2-ylmethyl, W═H, Y=—CH₂CH₂CH₂OMe, J=—CH₂CH₂OMe;
Compound of Formula (I), wherein M=-Ph, Q=4-tent-butyl-3-methoxyphenyl, Z=thiazol-2-ylmethyl, W═H, Y=—CH₂CH₂CH₂OMe, J=—CH₂CH₂NMe₂;
Compound of Formula (I), wherein M=-Ph, Q=4-tent-butyl-3-methoxyphenyl, Z=2-(thiazol-2-yl)ethyl, W═H, Y=—CH₂CH₂CH₂OMe, J=—CH₂CH₂NMe₂.

11. A pharmaceutical composition comprising a compound or a combination of compounds according to claim 1 or a pharmaceutically acceptable salt, stereoisomer, tautomer, prodrug, salt of a prodrug, or combination thereof, in combination with a pharmaceutically acceptable carrier or excipient.

12. A method of inhibiting the replication of an RNA-containing virus comprising contacting said virus with a therapeuctially effective amount of a compound or combination of compounds of claim 1, or a pharmaceutically acceptable salt, stereoisomer, tautomer, prodrug, salt of a prodrug, or combination thereof.

13. A method of treating or preventing infection caused by an RNA-containing virus comprising administering to a patient in need of such treatment a therapeutically effective amount of a compound or combination of compounds of claim 1, or a pharmaceutically acceptable salt, stereoisomer, tautomer, prodrug, salt of a prodrug, or combination thereof.

14. The method of claim 13 wherein the RNA-containing virus is hepatitis C virus.

15. The method of claim 13 further comprising the step of co-administering one or more agents selected from the group consisting of a host immune modulator and a second antiviral agent, or a combination thereof.

16. The method of claim 15 wherein the host immune modulator is selected from the group consisting of interferon-alpha, pegylated-interferon-alpha, interferon-beta, interferon-gamma, a cytokine, a vaccine and a vaccine comprising an antigen and an adjuvant.

17. The method of claim 15 wherein the second antiviral agent inhibits replication of HCV by inhibiting host cellular functions associated with viral replication.

18. The method of claim 15 wherein the second antiviral agent inhibits the replication of HCV by targeting proteins of the viral genome.

19. The method of claim 18 wherein said targeting protein is selected from the group consisting of helicase, protease, polymerase, metalloprotease, NS4A, NS4B, NS5A, and IRES.

20. The method of claim 13 further comprising the step of co-administering an agent or combination of agents that treat or alleviate symptoms of HCV infection including cirrhosis and inflammation of the liver.

21. The method of claim 13 further comprising the step of co-administering one or more agents that treat patients for disease caused by hepatitis B (HBV) infection.

22. The method of claim 13 further comprising the step of co-administering one or more agents that treat patients for disease caused by human immunodeficiency virus (HIV) infection.

23. A compound according to claim 1 selected from the group consisting of:
Compound of Formula (I), wherein M=2,4-difluorophenyl, Q=4-tent-butyl-3-methoxyphenyl, Z=thiazol-2-ylmethyl, W═H, Y=—CH₂CH₂CH₂OMe, J=1H-pyrazol-1-ylmethyl.
Compound of Formula (I), wherein M=tert-butyl, Q=4-tent-butyl-3-methoxyphenyl, Z=thiazol-2-ylmethyl, W═H, Y=—CH₂CH₂CH₂OMe, J=1H-pyrazol-1-ylmethyl.
Compound of Formula (I), wherein M=cyclopropyl, Q=4-tent-butyl-3-methoxyphenyl, Z=thiazol-2-ylmethyl, W═H, Y=—CH₂CH₂CH₂OMe, J=1H-pyrazol-1-ylmethyl.
Compound of Formula (I), wherein M=trifluoromethyl, Q=4-tent-butyl-3-methoxyphenyl, Z=thiazol-2-ylmethyl, W═H, Y=—CH₂CH₂CH₂OMe, J=1H-pyrazol-1-ylmethyl.
Compound of Formula (I), wherein M=2,6-difluorophenyl, Q=4-tent-butyl-3-methoxyphenyl, Z=thiazol-2-ylmethyl, W═H, Y=—CH₂CH₂CH₂OMe, J=1H-pyrazol-1-ylmethyl.
Compound of Formula (I), wherein M=2-chlorophenyl, Q=4-tent-butyl-3-methoxyphenyl, Z=thiazol-2-ylmethyl, W═H, Y=—CH₂CH₂CH₂OMe, J=1H-pyrazol-1-ylmethyl.
Compound of Formula (I), wherein M=2-cyanophenyl, Q=4-tent-butyl-3-methoxyphenyl, Z=thiazol-2-ylmethyl, W═H, Y=—CH₂CH₂CH₂OMe, J=1H-pyrazol-1-ylmethyl.
Compound of Formula (I), wherein M=2-trifluoromethylphenyl, Q=4-tent-butyl-3-methoxyphenyl, Z=thiazol-2-ylmethyl, W═H, Y=—CH₂CH₂CH₂OMe, J=1H-pyrazol-1-ylmethyl.
Compound of Formula (I), wherein M=2-trifluoromethoxyphenyl, Q=4-tent-butyl-3-methoxyphenyl, Z=thiazol-2-ylmethyl, W═H, Y=—CH₂CH₂CH₂OMe, J=1H-pyrazol-1-ylmethyl.
Compound of Formula (I), wherein M=2-fluorophenyl, Q=4-tert-butyl-2-fluoro-3-methoxyphenyl, Z=thiazol-2-ylmethyl, W═H, Y=—CH₂CH₂CH₂OMe, J=1H-pyrazol-1-ylmethyl.
Compound of Formula (I), wherein M=2-fluorophenyl, Q=4-tert-butyl-2,6-difluoro-3-methoxyphenyl, Z=thiazol-2-ylmethyl, W═H, Y=—CH₂CH₂CH₂OMe, J=1H-pyrazol-1-ylmethyl.
Compound of Formula (I), wherein M=methyl, Q=4-tent-butyl-3-methoxyphenyl, Z=pyridin-2-ylmethyl, W═H, Y=—CH₂CH₂CN, J=1H-pyrazol-1-ylmethyl.
Compound of Formula (I), wherein M=methyl, Q=4-tent-butyl-3-methoxyphenyl, Z=(5-methylisoxazol-3-yl)methyl, W═H, Y=—CH₂CH₂CN, J=1H-pyrazol-1-ylmethyl.
Compound of Formula (Ia), wherein M=2-fluorophenyl, Q=5-bromo-4-tert-butyl-2-fluorophenyl, Z=pyridin-2-ylmethyl, W═H, Y=—CH₂CH₂CH₂OMe, J=1H-pyrazol-1-ylmethyl.
Compound of Formula (I), wherein M=Me, Q=3-bromo-4-tert-butylphenyl, Z=benzyl, W═H, Y=—CH₂CH₂CH₂OMe, J=1H-pyrazol-1-ylmethyl.
Compound of Formula (I), wherein M=2-thiophenyl, Q=4-tert-butyl-3-methoxyphenyl, Z=thiazol-2-ylmethyl, W═H, Y=—CH₂CH₂CH₂OMe, J=1H-pyrazol-1-ylmethyl.
Compound of Formula (I), wherein M=phenyl, Q=3-bromo-4-tert-butylphenyl, Z=thiazol-2-ylmethyl, W═H, Y=—CH₂CH₂CN, J=1H-pyrazol-1-ylmethyl.
Compound of Formula (I), wherein M=phenyl, Q=naphthalen-2-yl, Z=thiazol-2-ylmethyl, W═H, Y=—CH₂CH₂CN, J=1H-pyrazol-1-ylmethyl.
Compound of Formula (I), wherein M=phenyl, Q=4-tert-butyl-3-difluoromethylphenyl, Z=thiazol-2-ylmethyl, W═H, Y=—CH₂CH₂CN, J=1H-pyrazol-1-ylmethyl.
Compound of Formula (I), wherein M=phenyl, Q=4-tert-butyl-3-(1,1-difluoroethyl)phenyl, Z=thiazol-2-ylmethyl, W═H, Y=—CH₂CH₂CN, J=1H-pyrazol-1-ylmethyl.
Compound of Formula (I), wherein M=phenyl, Q=4-tent-butyl-3-difluoromethoxyphenyl, Z=thiazol-2-ylmethyl, W═H, Y=—CH₂CH₂CN, J=1H-pyrazol-1-ylmethyl.
Compound of Formula (I), wherein M=phenyl, Q=6-bromo-4-tert-butyl-3-methoxyphenyl, Z=thiazol-2-ylmethyl, W═H, Y=—CH₂CH₂CN, J=1H-pyrazol-1-ylmethyl.
Compound of Formula (I), wherein M=2-fluorophenyl, Q=4-tent-butyl-3-fluorophenyl, Z=thiazol-2-ylmethyl, W═H, Y=—CH₂CH₂CH₂OMe, J=1H-pyrazol-1-ylmethyl.
Compound of Formula (I), wherein M=2-fluorophenyl, Q=4-tert-butyl-3-chlorophenyl, Z=thiazol-2-ylmethyl, W═H, Y=—CH₂CH₂CH₂OMe, J=1H-pyrazol-1-ylmethyl.
Compound of Formula (I), wherein M=2-fluorophenyl, Q=4-tert-butyl-6-fluoro-3-methoxyphenyl, Z=thiazol-2-ylmethyl, W═H, Y=—CH₂CH₂CH₂OMe, J=1H-pyrazol-1-ylmethyl.
Compound of Formula (I), wherein M=2-fluorophenyl, Q=7-tert-butyl-benzoxazol-4-yl, Z=thiazol-2-ylmethyl, W═H, Y=—CH₂CH₂CH₂OMe, J=1H-pyrazol-1-ylmethyl.
Compound of Formula (I), wherein M=2-fluorophenyl, Q=4-tert-butyl-3-ethoxylphenyl, Z=thiazol-2-ylmethyl, W═H, Y=—CH₂CH₂CH₂OMe, J=1H-pyrazol-1-ylmethyl.
Compound of Formula (Ia), wherein M=2-fluorophenyl, Q=4-tert-butyl-6-fluoro-3-methoxyphenyl, Z=thiazol-2-ylmethyl, W═H, Y=—CH₂CH₂CH₂OMe, J=1H-pyrazol-1-ylmethyl.
The Opposite Enantiomer of Compound of Formula (Ia), wherein M=2-fluorophenyl, Q=4-tent-butyl-6-fluoro-3-methoxyphenyl, Z=thiazol-2-ylmethyl, W═H, Y=—CH₂CH₂CH₂OMe, J=1H-pyrazol-1-ylmethyl.
Compound of Formula (Ia), wherein M=2-fluorophenyl, Q=3-bromo-4-tert-butyl-6-fluorophenyl, Z=thiazol-2-ylmethyl, W═H, Y=—CH₂CH₂CH₂OMe, J=1H-pyrazol-1-ylmethyl.
Compound of Formula (Ia), wherein M=2-fluorophenyl, Q=3-bromo-4-tert-butyl-2-fluorophenyl, Z=thiazol-2-ylmethyl, W═H, Y=—CH₂CH₂CH₂OMe, J=1H-pyrazol-1-ylmethyl.
Compound of Formula (Ia), wherein M=2-fluorophenyl, Q=3-bromo-4-tert-butylphenyl, Z=thiazol-2-ylmethyl, W═H, Y=—CH₂CH₂CH₂OMe, J=1H-pyrazol-1-ylmethyl.
Compound of Formula (Ia), wherein M=2-fluorophenyl, Q=4-tent-butyl-3-methoxyphenyl, Z=thiazol-2-ylmethyl, W═H, Y=—CH₂CH₂CH₂OMe, J=1H-pyrazol-1-ylmethyl.

24. The composition of claim 11, further comprising a cytochrome P450 monooxygenase inhibitor or a pharmaceutically acceptable salt thereof.

25. The composition of claim 24, wherein the cytochrome P450 mooxygenase inhibitor is ritonavir.
